(19) United States
(12) Patent Application Publication
(10) Pub. No.: US 2015/0112324 A1
(43) Pub. Date: Apr. 23, 2015

(54) RF TISSUE MODULATION DEVICES AND METHODS OF USING THE SAME

(71) Applicant: Trice Medical, Inc., Wilmington, DE (US)

(72) Inventor: James S. Cybulski, Menlo Park, CA (US)

(21) Appl. No.: 14/526,289

(22) Filed: Oct. 28, 2014

Related U.S. Application Data
(63) Continuation of application No. 13/085,355, filed on Apr. 12, 2011, now abandoned.
(60) Provisional application No. 61/323,269, filed on Apr. 12, 2010.

(51) Int. Cl. A61B 18/04; A61B 18/12

(52) U.S. Cl. CPC A61B 18/042; A61B 18/1206; A61B 2018/1226; A61B 2018/1266; A61B 2018/1273
USPC 606/34

(57) ABSTRACT

Minimally invasive RF tissue modulation devices are provided. In some aspects, the devices include a hand-held control unit and an elongated member. The hand-held control unit includes an electrical energy source and the elongated member has a proximal end operably coupling to the hand-held control unit. The RF tissue modulation device is configured to generate a plasma at a distal end plasma generator for a therapeutic duration. In some aspects, RF tissue modulation devices are provided and include an adapter that operably couples to a hand-held medical device. The adapter generates RF energy for delivery to a plasma generator on an elongated member. Methods of delivering the RF energy to the internal target tissue site are also provided.

### CROSS-REFERENCE TO RELATED APPLICATIONS

This application is a continuation of U.S. patent application Ser. No. 13/085,355, filed on Apr. 12, 2011, entitled “RF TISSUE MODULATION DEVICES AND METHODS OF USING THE SAME,” which claims the benefit of U.S. Provisional Application No. 61/323,269, filed on Apr. 12, 2010, entitled “RF TISSUE MODULATION DEVICES AND METHODS OF USING THE SAME.” The contents of the aforementioned applications are hereby incorporated by reference in their entireties as if fully set forth herein. The benefit of priority to the foregoing applications is claimed under the appropriate legal basis, including, without limitation, under 35 U.S.C. §119(e).

### BACKGROUND OF THE INVENTION

Traditional surgical procedures, both therapeutic and diagnostic, for pathologies located within the body can cause significant trauma to the intervening tissues. These procedures often require a long incision, extensive muscle stripping, prolonged retraction of tissues, denervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues.

### SUMMARY OF THE INVENTION

Minimally invasive RF tissue modulation devices are provided. Aspects of the devices include a hand-held control unit and an elongated member. The hand-held control unit includes an electrical energy source and the elongated member has a proximal end operably coupled to the hand-held control unit. A distal end of the elongated member includes a plasma generator. The minimally invasive RF tissue modulation device is configured to generate a plasma at the plasma generator for a therapeutic duration.

An adapter is also provided. Aspects of the invention include an adapter having an electrical energy source, voltage converter, charge accumulator, and RF signal generator.

An RF probe is also provided. Aspects of the RF probe include an elongated member configured to operably couple to a hand-held device at a proximal end of the elongated member. Furthermore, the minimally-dimensioned distal end of the elongated member includes a plasma generator.

A hand-held minimally dimensioned device configured to operably couple to an adapter and an RF probe, such as the ones described above, is also provided. Also provided are kits including a set of components selected from a group consisting of a hand-held device, adapter, RF probe, and other types of probes such as a visualization probe, as described above.

Also provided are methods of delivering the RF energy to the internal target tissue site are also provided. The methods include positioning the distal end of an elongated member of a device, such as the minimally invasive RF tissue modulation device described above, at the internal target tissue site of a subject. The methods also include generating a plasma from the plasma generator to deliver RF energy to the internal target tissue site of the subject.

### BRIEF DESCRIPTION OF THE DRAWINGS

FIG. 1A is a side view of one embodiment of a RF tissue modulation device including a elongated member and hand-held control unit.

FIG. 1B is a perspective view of the RF tissue modulation device of FIG. 1A.

FIG. 2A is a cross sectional side view of the distal end of the elongated member of RF tissue modulation device, according to one embodiment.

FIG. 2B is a cross sectional side view of the distal end of the elongated member of RF tissue modulation device, according to one embodiment.

FIG. 2C is a cross sectional side view of the distal end of the elongated member of RF tissue modulation device, according to one embodiment.

FIG. 2D is a cross sectional side view of the distal end of the elongated member of RF tissue modulation device, according to one embodiment.

FIG. 2E is a cross sectional side view of the distal end of the elongated member of an RF tissue modulation device, according to one embodiment.

FIGS. 3A and 3B are side views of an adapter operably coupled to a medical device, according to two different embodiments.

FIG. 4A is a side view of a medical device separated from an adapter configured to operably couple to the medical device, according to one embodiment.

FIG. 4B is a side view of the separated medical device and adapter of FIG. 3A with a removable section of the medical device removed, according to one embodiment.

FIG. 4C is a side view of the adapter and medical device of FIG. 3A operably coupled, according to one embodiment.

FIG. 5 is a side view of an adapter operably coupled to a medical device, according to one embodiment.

FIG. 6 is a functional block diagram of an RF energy source, according to one embodiment.

FIG. 7 is a functional block diagram of an RF energy source, according to one embodiment.

FIG. 8 is a functional block diagram of an RF energy source, according to one embodiment.

FIG. 9 is a block diagram showing an embodiment of the electrical energy source and voltage converter shown for the RF energy source of FIG. 8.

FIG. 10 is a block diagram showing an embodiment of the charge accumulator shown for the RF energy source of FIG. 8.

FIG. 11 is a block diagram showing an embodiment of a modulation circuit coupled to the charge accumulator shown for the RF energy source of FIG. 8.

FIG. 12 is a block diagram showing an embodiment of an RF signal generator and RF tuner shown for the RF energy source of FIG. 8.

### DETAILED DESCRIPTION OF THE PREFERRED EMBODIMENTS

Minimally invasive RF tissue modulation devices are provided. Aspects of the devices include a hand-held control unit and an elongated member. The hand-held control unit includes an electrical energy source and the elongated member has a proximal end operably coupled to the hand-held control unit. A distal end of the elongated member includes a plasma generator. The minimally invasive RF tissue modulation device is configured to generate a plasma at the plasma generator for a therapeutic duration.

An adapter is also provided. Aspects of the invention include an adapter having an electrical energy source, voltage converter, charge accumulator, and RF signal generator.

An RF probe is also provided. Aspects of the RF probe include an elongated member configured to operably couple to a hand-held device at a proximal end of the elongated member. Furthermore, the minimally-dimensioned distal end of the elongated member includes a plasma generator.

A hand-held minimally dimensioned device configured to operably couple to an adapter and an RF probe, such as the ones described above, is also provided. Also provided are kits including a set of components selected from a group consisting of a hand-held device, adapter, RF probe, and other types of probes such as a visualization probe, as described above.

Also provided are methods of delivering the RF energy to the internal target tissue site are also provided. The methods include positioning the distal end of an elongated member of a device, such as the minimally invasive RF tissue modulation device described above, at the internal target tissue site of a subject. The methods also include generating a plasma from the plasma generator to deliver RF energy to the internal target tissue site of the subject.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, aspects of embodiments of the subject RF tissue modulation devices are described first in greater detail. Next, embodiments of methods of modifying, and in some instances additionally visualizing, an internal target tissue of a subject in which the subject RF tissue modulation devices may find use are reviewed in greater detail.

### RF Tissue Modulation Devices

As summarized above, RF tissue modulation devices of the invention may include an elongated member and a hand-held control unit (such as an RF probe and hand-held control unit described further below). For example, the elongated member may be operably coupled to the hand-held device at a proximal end of the elongated member. In other aspects of the invention, RF tissue modulation devices may include an elongated member and an adapter configured to be independently removably coupled to a medical device (e.g., a visualization device). In some instances, the elongated member removably couples to the medical device. It should also be understood, that that in some instances, the elongated member may be affixed to the medical device, adapter, and/or hand-held control unit. Furthermore, it should be understood that the term RF tissue modulation devices is used herein to refer generally to cumulative devices (e.g., RF probe and hand-held device; or, RF probe, adapter, and medical device), and in some instances to refer to each of the individual or combination of component devices (e.g., RF probe, or adapter, or RF probe and adapter, etc.).

In addition to the above two components, devices of certain embodiments of the invention may include an RF energy source that is configured to generate a plasma at the plasma generator of the elongated member (e.g., RF probe) for a therapeutic duration, e.g., as described above. The RF energy source may include a number of distinct components, such as but not limited to: an electrical energy source, voltage converter, charge accumulator, and RF signal generator. In certain instances, the devices may also include an adaptor, as described in greater detail below. The various components of the RF energy source may be present in one of the handheld control unit or adaptor (when present) or distributed among the various components of the device, e.g., the hand held control unit, adaptor and/or RF probe.

### RF Probe

The RF probe is an elongated member that is configured to be operably coupled to a hand-held control unit. With respect to the elongated member, this component has a length that is 1.5 times or longer than its width, such as 2 times or longer than its width, including 5 or even 10 times or longer than its width, e.g., 20 times longer than its width, 30 times longer than its width, or longer. The length of the elongated member may vary, and in some instances ranges from 5 cm to 20 cm, such as 7.5 cm to 15 cm and including 10 to 12 cm. The elongated member may have the same outer cross sectional dimensions (e.g., diameter) along its entire length. Alternatively, the cross sectional diameter may vary along the length of the elongated member.

In some instances, at least the distal end region of the elongated member of the device is dimensioned to pass through a Cambin's triangle. By distal end region is meant a length of the elongated member starting at the distal end of 1 cm or longer, such as 3 cm or longer, including 5 cm or longer, where the elongated member may have the same outer diameter along its entire length. The Cambin's triangle (also known in the art as the Pambin's triangle) is an anatomical spinal structure bounded by an exiting nerve root and a traversing nerve root and a disc. The exiting root is the root that leaves the spinal canal just cephalad (above) the disc, and the traversing root is the root that leaves the spinal canal just caudad (below) the disc. Where the distal end of the elongated member is dimensioned to pass through a Cambin's triangle, at least the distal end of the device has a longest cross sectional dimension that is 10 mm or less, such as 8 mm or less and including 7 mm or less. In some instances, the devices include an elongated member that has an outer diameter at least in its distal end region that is 5.0 mm or less, such as 4.0 mm or less, including 3.0 mm or less.

The elongated members of the subject RF tissue modulation devices have a proximal end and a distal end. The term “proximal end”, as used herein, refers to the end of the elongated member that is nearer the user (such as a physician operating the device in a tissue modification procedure), and the term “distal end”, as used herein, refers to the end of the elongated member that is nearer the internal target tissue of the subject during use. The proximal end is also the end that is operably coupled to the hand-held control unit of the device (described in greater detail below). The elongated member is, in some instances, a structure of sufficient rigidity to allow the distal end to be pushed through tissue when sufficient force is applied to the proximal end of the elongate member. As such, in these embodiments the elongated member is not pliant or flexible, at least not to any significant extent.

As summarized above, some embodiments of the RF tissue modulation devices include a plasma generator integrated at the distal end of the elongated member, such that the plasma generator is integrated with the elongated member. As the plasma generator is integrated at the distal end of the device, it cannot entirely be removed from the remainder of the device without significantly compromising the structure and functionality of the device. While the plasma generator cannot entirely be removed from the device without compromising the structure and functionality of the device, components of the plasma generator may be removable and replaceable. For example, an RF electrode of a plasma generator according to some embodiments may be configured such that a wire component of the plasma generator may be replaceable while the remainder of the plasma generator is not. Accordingly, the devices of the present invention are distinguished from devices which include a “working channel” through which a separate autonomous plasma generator device, such as autonomous RF electrode device, is passed through. In contrast to such devices, since the plasma generator of the present device is integrated at the distal end, it is not a separate device from the elongated member that is merely present in a working channel of the elongated member and which can be removed from the working channel of such an elongated member without structurally compromising the elongated member in any way. The plasma generator may be integrated with the distal end of the elongated member by a variety of different configurations. Integrated configurations include configurations where the plasma generator is fixed relative to the distal end of the elongated member, as well as configurations where the plasma generator is movable to some extent relative to the distal end of the elongated member may be employed in devices of the invention. Specific configurations of interest are further described below in connection with the figures. As the plasma generator is a distal end integrated plasma generator, it is located at or near the distal end of the elongated member. Accordingly, it is positioned at 30 mm or closer to the distal end, such as at 20 mm or closer to the distal end, including at 10 mm or closer to the distal end. In some instances, the plasma generator is located at the distal end of the elongated member.

The plasma generator may be configured in a variety of ways for a controllable delivery of RF energy. The plasma generator may include one or more RF electrodes positioned at the distal end of the elongated member. RF electrodes are devices for the delivery of radiofrequency (RF). In some instances, the RF electrodes are electrical conductors, such as a metal wire, or other conductive member, and can be dimensioned to access an intervertebral disc space for example.

RF electrodes may be shaped in a variety of different formats, such as circular, square, rectangular, oval, etc. The dimensions of such electrodes may vary, where in some embodiments the RF electrode has a longest cross sectional dimension that is 7 mm or less, 6 mm or less 5 mm or less, 4 mm or less, 3 mm or less or event 2 mm or less, as desired. Where the RF electrode includes a wire, the diameter of the wire in such embodiments may be 180 μm, such as 150 μm or less, such as 130 μm or less, such as 100 μm or less, such as 80 μm or less.

Various RF electrode configurations for use in tissue modification devices are described in U.S. Pat. Nos. 7,449,019; 7,137,981; 6,997,941; 6,837,887; 6,241,727; 6,112,123; 6,607,529; 5,334, 183; in Provisional Application Ser. No. 61/082,774; in U.S. patent application Ser. No. 12/422,176; and in International patent application Ser. No. 09/51446; the disclosures of which are herein incorporated by reference. RF electrode systems or components thereof may be adapted for use in devices of the present invention (when coupled with guidance provided by the present specification) and, as such, the disclosures of the RF electrode configurations in these patents are herein incorporated by reference. Specific RF electrode configurations of interest are further described in connection with the figures, below.

In some aspects of the invention, the plasma generator is configured to generate a plasma between two or more RF electrodes. In some instances, one or more of the RF electrodes is a grounded conductive member, wherein a plasma is generated between an RF electrode and a grounded RF electrode (e.g., grounded conductive member, such as grounded outer surface of the elongated member, etc.). The RF electrodes are provided with the necessary power and voltage to generate a plasma between the electrodes. In some instances, the plasma is only generated when the plasma generator is partially or fully submerged in saline solution such that only a portion of the plasma field is exposed to the patient. The surrounding saline solution provides a conductive path between the electrodes as well as the sodium ions required to produce the plasma. The saline solution may also help to disperse the thermal effects generated by the plasma field. Such limited exposure may also help to confine the treated region to the surface surrounding tissue. In some instances, the plasma may be generated in other mediums, such as air, blood, tissue, etc.

RF electrodes may be positioned in a variety of ways at the distal end of the elongated member. For example, one or more RF electrodes may be positioned on the elongated member, extending from the elongated member, and/or positioned within the elongated member. In some instances, the plasma generator is configured to produce a plasma between an RF electrode positioned inside of the distal end of the elongated member and an outer surface of the elongated member. In some instances, the plasma generator may be configured to produce a plasma between an RF electrode positioned substantially at a tip of the elongated member and the outer surface of the elongated member. In this way, the tip of the elongated member may correspond approximately to the target tissue site.

The position of the RF electrodes may depend on specific application and design considerations (e.g., field of view of the user holding the device, and/or positioning of other components in the elongated member (e.g., visualization sensor, illuminator, etc.). For example, in some instances, the elongated member may include a distal end integrated visualization sensor in addition to the plasma generator, and the hand-held device further include a monitor, such as described in further detail below.

The elongated member may also include an opening positioned at the distal end of the elongated member. The opening may be of a variety of shapes—e.g., oval, circular, rectangular, open-ended, etc.). The size of the opening may vary depending on particular application and design considerations. Example opening sizes may include, for example, 20 mm or less, such as 10 mm or less and including 5 mm or less, e.g., 2.5 mm or less. In some embodiments, the elongated member may include an opening positioned over a conductive member acting as an RF electrode positioned within the distal end of the elongated member. In another embodiment, the elongated member may include a conductive member acting as an RF electrode positioned within or near the opening at the distal end of the elongated member.

In some instances, the elongated member includes one or more insulators coupled to one or more RF electrodes. The insulator may be of a variety of materials, such as ceramic, or any other insulative material. The insulators may be used to maintain the RF electrodes in position. For example, an insulator may be positioned in the elongated member to maintain an RF electrode (e.g., conductive member such as a small metal wire or plate) within the elongated member. Multiple insulators may be positioned within or on the elongated member to maintain one or more RF electrodes within or near an opening at the distal end of the elongated member.

In some aspects of the invention, the plasma generator receives an RF signal generated by an RF energy source. The plasma generator is supplied with current and the voltage signal driving the current to the plasma generator may be definable as a sine, square, saw-tooth, triangle, pulse, non-standard, complex, or irregular waveform, or the like, with a well-defined operating frequency. For example, the operating frequency can range from 1 KHz to 50 MHz, such as from 100 KHz to 25 MHz, and including from 250 KHz to 10 MHz. Furthermore, the operating frequency can be modulated by a modulation waveform. By “modulated” is meant attenuated in amplitude by a second waveform, such as a periodic signal waveform. The modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 70 Hz. Thus, in some instances, the plasma generator receives a high voltage modulated RF signal and generates a plasma.

In some aspects of the invention, an RF line may couple one or more RF electrodes described above to an RF energy source. The RF line may be made of any conductive material, such as metal, metal alloys, etc. The RF line electrically couples the plasma generator to the RF energy source at another location of the device, such as a proximal end location. Such proximal end location may include, for example, a hand-held control unit or adapter as described in further detail below. The RF line may be positioned, for example, within or along the elongated member to couple the proximal end RF energy source to the distal end plasma generator.

The RF tissue modulation device may be configured to deliver RF energy to the plasma generator for a therapeutic duration. The therapeutic duration may last, for example, minutes or less, such as 1 minute or less, including 30 seconds or less, such as 10 seconds or less. In some instances, the therapeutic duration may range from 1 to 2 seconds. Visualization capabilities (as developed in greater detail below), if implemented, may be available for a duration independent of the therapeutic duration. For instance, visualization capabilities may continue after RF energy treatment.

In some instances, an RF shield is positioned within the elongated member adjacent to the RF line in order to provide RF shielding for the ambient RF field generated. The RF shielding is positioned in the elongated member so as to minimize ambient RF interference and disturbances encountered by other components in the device (e.g., visualization sensors, chips, etc.). The term “adjacent to” herein is meant to include next to, surrounding, or substantially next to or surrounding. For example, RF shielding may be provided around the RF line and/or substantially around the RF electrodes. In some instances, RF shielding may be provided substantially around or near other components which require protection from ambient RF. In some instances, the RF shielding is provided between the components which require protection and the RF line (and/or RF electrodes) but not necessarily around them.

### RF Energy Source

In some aspects of the invention, embodiments of the RF tissue modulation devices include an RF energy source used to generate RF energy for delivery to the plasma generator. For example, the RF energy source may generate a high voltage modulated RF signal for delivery to the plasma generator. The RF energy source may include, for example, an electrical energy source, a voltage converter, charge accumulator, and a RF signal generator. In some instances, the voltage converter, charge accumulator, and RF signal generator operably couple the electrical energy source to a plasma generator on an elongated member (e.g., RF probe).

In some aspects of the invention, the RF energy source is included in a hand-held control unit. In some instances, the hand-held control unit may be a hand-held medical device (such as, for example, a tissue visualization device as described in U.S. application Ser. No. 12/501,336, the disclosure of which is hereby incorporated by reference) that has been configured to further include an RF energy source. In some aspects of the invention, the RF energy source is included in an adapter configured to removably couple to a hand-held device, such as a hand-held medical device, such as a tissue visualization device as described in U.S. application Ser. No. 12/501,336, the disclosure of which is hereby incorporated by reference.

The electrical energy source may include one or more power sources—e.g., one or more DC batteries. While the electrical energy source is described as being located within the hand-held control unit or adapter, in some instances, the electrical energy source may be remote from the hand-held control unit or adapter—e.g., in a battery pack configured to be electrically coupled to the hand-held control unit or adapter—e.g., via cables. However, providing the electrical energy source within the hand-held control unit or adapter allows the RF tissue modulation device to remain untethered and more portable, which may be user-friendly for the operator of the device.

The charge accumulator stores electrical energy which is later discharged when RF energy is to be delivered to the plasma generator. The charge accumulator may be, for example, one or more capacitors that charge until delivery of RF energy is activated by the user. In some instances, the charge accumulator is coupled to an electrical energy source and stores energy in one or more capacitors until RF energy is activated. To activate the RF energy, the user may engage a switch or other activation element, such as a button, key, wheel, trigger, etc., which initiates the decoupling of the charge accumulator from the electrical energy source so that it may begin discharging. The one or more capacitors may be selected to provide most the current, so that less current is required by the electrical energy source. In some instances, this configuration provides a large current in a short amount of time. Further, the one or more capacitors may be chosen, for example, to have less impedance than the internal resistance of the DC batteries.

In some instances, the charge accumulator may be configured to receive a voltage signal from a component other than the electrical energy source. For example, the charge accumulator may be coupled to the voltage converter and receive a high voltage signal which charges the charge accumulator. When RF energy is activated, the voltage converter is disconnected from the charge accumulator, for example, to provide for discharge.

In some instances, the charge accumulator may include two or more capacitor pairs which may be discharged sequentially in stages. For example, each pair of capacitors may be configured to provide a respective positive and negative voltage output. In some instance, a modulation circuit may be configured to couple to the charge accumulator and discharge the two or more capacitor pairs sequentially at a modulated rate based on a clock signal from a clock source. For example, the modulation circuit may include a demultiplexer configured to receive a count from a counter and to discharge stages of the charge accumulator based on the count. The counter may be configured to count at a rate based on the clock signal from the clock source and discharge each stage on an associated count. The modulation circuit may further include a timer coupled to the enable input of the demultiplexer, for example, to activate the discharging of the capacitor pairs when RF energy is activated. Upon completion of the timer count, the timer disables the demultiplexer so that the capacitor pairs are no longer triggered to discharge and may once again charge.

The voltage converter receives an input signal at a first voltage level and generates an output signal at a second voltage level. Voltage converters may include, for example, a DC to DC converter, transformer, etc. The voltage converter boosts the voltage level and generates a high voltage signal necessary for plasma generation. While it should be understood that a voltage boost is not necessarily required if the electrical energy source provides sufficient voltage, in typical applications, practical design considerations (e.g., weight and size) limit the batteries to a voltage level which requires further boosting.

In some embodiments, the voltage booster is configured to receive a modulated RF signal and to output a high voltage modulated RF signal. In some embodiments, the voltage converter is configured to receive a DC voltage signal from the charge accumulator and to output a high voltage signal (e.g., to an RF signal generator). In some instances, the voltage converter may further be configured to receive a clock signal from a clock source, in addition to a voltage signal, and to output a modulated high voltage signal based on the clock signal. In some instances, the voltage converter may include more than one DC to DC converter and be configured to generate a positive and negative high voltage rail with a common ground.

The RF signal generator generates an RF signal at a desired operating frequency to provide the necessary power delivery to the plasma generator. The RF signal may be in the form of, for example, a sine, square, saw-tooth, triangle, pulse, non-standard, complex, or irregular waveform, or the like, with a well-defined operating frequency. For example, the operating frequency can range from 1 KHz to 50 MHz, such as from 100 KHz to 25 MHz, and including from 250 KHz to 10 MHz. In some embodiments, the RF voltage signal is a sine wave with operating frequency 460 kHz.

In some embodiments, the RF signal generator includes an RF power amplifier and an RF clock source. The RF power amplifier receives an RF clock signal generated by the RF clock source and generates an RF signal at an operating frequency based on the RF clock signal. In some instances, the RF power amplifier may be configured to receive a voltage signal used as a bias voltage input. The bias voltage input may affect, for example, the peak voltage of the signal output by the RF power amplifier. The bias voltage signal may be received by another component such as the charge accumulator, DC to DC converter, or other voltage source. For example, in some embodiments, the RF signal generator is configured to receive a bias voltage signal from a charge accumulator, as well as receive an RF clock signal from an RF clock source, and to output an RF signal based on the bias voltage signal and RF clock signal.

In some embodiments, the RF power amplifier is configured to receive a second clock signal from a second clock source and generate a modulated RF output signal based on the second clock signal. By “modulated” it is meant that the modulation frequency comprises attenuating the amplitude of the RF signal based on the second clock signal. The modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 50 Hz.

In some instances, the RF power amplifier is configured to receive a modulated bias voltage signal (e.g., from another component such as a DC to DC converter, or other voltage converter), as well as an RF clock signal from an RF clock source, and to output a modulated RF signal—e.g., the RF signal is based on the RF clock signal and is modulated based on the modulated bias voltage signal. For example, the voltage converter may be coupled to a clock source and receive a clock signal and provide a high voltage modulated output signal based on the clock signal to the RF power amplifier.

In some embodiments, the RF signal generator comprises an H-bridge. In some instances, the H-bridge is coupled to an RF clock source and configured to receive an RF clock signal from the RF clock source and operate at a frequency based on the RF clock signal. For instance, the H-bridge may receive positive and negative voltage input signals and generate positive and negative voltage output signals that switch polarities at an operating frequency based on the RF clock signal.

In some instances, the RF energy source may include a bandpass filter to filter out out-of-band frequencies. Any convenient bandpass filter may be employed.

The RF energy source may also include an RF tuner in some embodiments. The RF tuner includes basic electrical elements (e.g., capacitors and inductors) which serve to tailor the output impedance of the RF energy system. The term “tailor” is intended here to have a broad interpretation, including affecting an electrical response that achieves maximum power delivery, affecting an electrical response that achieves constant power (or voltage) level under different loading conditions, affecting an electrical response that achieves different power (or voltage) levels under different loading conditions, etc. Furthermore, the elements of the RF tuner can be chosen so that the output impedance is dynamically tailored, meaning the RF tuner self-adjusts according to the load impedance encountered at the electrode tip. For instance, the elements may be selected so that the electrode has adequate voltage to develop a plasma corona when the electrode is placed in a saline solution (with saline solution grounded to return electrode), but then may self-adjust the voltage level to a lower threshold when the electrode contacts tissue (with tissue also grounded to return electrode, for example through the saline solution), thus dynamically maintaining the plasma corona at the electrode tip while minimizing the power delivered to the tissue and the thermal impact to surrounding tissue. RF tuners, when present, can provide a number of advantages. For example, delivering RF energy to target tissue through the distal tip of the electrode is challenging since RF energy experiences attenuation and reflection along the length of the conductive path from the RF energy source to the electrode tip, which can result in insertion loss. Inclusion of an RF tuner, e.g., as described above, can help to minimize and control insertion loss.

The RF tissue modulation devices may be configured to deliver RF energy from the RF energy source to the plasma generator for a therapeutic duration. The therapeutic duration may range, for example, from minutes or less, such as 1 minute or less, including 30 seconds or less, such as 10 seconds or less. In some instances, the therapeutic duration may range from 1 to 2 seconds

Furthermore, the RF tissue modulation device may be configured to recharge the charge accumulator within a minimum recharge period between plasma generation. The minimum recharge period may range, for example, from 10 minutes or less, including 5 minutes or less, such as 3 minutes or less. In some instances, the minimum recharge period ranges from 1 to 2 minutes.

### Hand-Held Control Unit

As summarized above, the RF tissue modulation devices of the invention further include a hand-held control unit to which the elongated member is operably connected. By “operably connected” is meant that one structure is in communication (for example, mechanical, electrical, optical connection, or the like) with another structure. The hand-held control unit is located at the proximal end of the elongated structure. As the control unit is hand-held, it is configured to be held easily in the hand of an adult human. Accordingly, the hand-held control unit may have a configuration that is amenable to gripping by the human adult hand. The weight of the hand-held control unit may vary, but in some instances is 10 lbs or less, including 5 lbs or less, such 3 lbs or less. In some instances, the weight of the hand-held control may weigh 2 lbs or less, including 1 lb or less. The hand-held control unit may have any convenient configuration, such as a hand-held wand with one or more control buttons, as a hand-held gun with a trigger, etc.

In some aspects of the invention, the hand-held control unit includes the RF energy source. For example, the hand-held control unit may include an electrical energy source, a charge accumulator, voltage converter, and RF signal generator, wherein the voltage converter, charge accumulator, and RF signal generator operably couple the electrical energy source to a plasma generator on an elongated member (e.g., RF probe). In some instances, the RF energy source may additionally include a bandpass filter and/or RF tuner. In some instances, the bandpass filter and/or RF tuner are located external to the hand-held control unit—e.g., in the elongated member.

In some instances, the hand-held control unit may include a monitor. By monitor is meant a visual display unit, which includes a screen that displays visual data in the form of images and/or text to a user. The screen may vary, where a screen type of interest is an LCD screen. The monitor, when present, may be integrated or detachable from the remainder of the hand-held control unit. As such, in some instances the monitor may be an integrated structure with the hand-held control unit, such that it cannot be separated from the hand-held control unit without damaging the monitor in some manner. In yet other embodiments, the monitor may be a detachable monitor, where the monitor can be attached to and separated from the hand-held control unit, as desired, without damaging the function of the monitor. In such embodiments, the monitor and hand-held control unit may have a variety of different mating configurations, such as where the hand-held control unit includes a hole configured to receive a post of the monitor, where the monitor has a structure that is configured to snap onto a receiving structure of the hand-held control unit, etc. The monitor, when present will have dimensions sufficient for use with the hand-held control unit, where screen sizes of interest may include 10 inches or smaller, such as 8 inches or smaller, e.g., 5 inches or smaller, e.g., 3.5 inches, etc.

Data communication between the monitor and the remainder of the hand-held control unit may be accomplished according to any convenient configuration. For example, the monitor and remaining components of the hand-held control unit may be connected by one or more wires. Alternatively, the two components may be configured to communication with each other via a wireless communication protocol. In these embodiments, the monitor will include a wireless communication module.

In some embodiments, the distal end of the elongated member is rotatable about its longitudinal axis when a significant portion of the hand-held control unit is maintained in a fixed position. As such, at least the distal end of the elongated member can turn by some degree while the hand-held control unit attached to the proximal end of the elongated member stays in a fixed position. The degree of rotation in a given device may vary, and may range from 0 to 360°, such as 0 to 270°, including 0 to 180°. Rotation, when present, may be provided by any convenient approach, e.g., through use of motors.

Devices of the invention may be disposable or reusable. As such, devices of the invention may be entirely reusable (e.g., be multi-use devices) or be entirely disposable (e.g., where all components of the device are single-use). In some instances, the device can be entirely reposable (e.g., where all components can be reused a limited number of times). Each of the components of the device may individually be single-use, of limited reusability, or indefinitely reusable, resulting in an overall device or system comprised of components having differing usability parameters.

Of interest are devices in which the hand-held control unit is reusable. In such devices, the elongated member is configured to be detachable from the hand-held control unit. As the elongated member is configured to be readily separable from the hand-held control unit without in any way damaging the functionality of the hand-held control unit, such that the hand-held control unit may be attached to another elongated member. As such, the devices are configured so that the hand-held control unit can be sequentially operably attached to multiple different elongated members. Of interest are configurations in which the elongated member can be manually operably attached to a hand-held control unit without the use of any tools. A variety of different configurations may be employed, e.g., where the proximal end of the elongated member engages the hand-held control unit to provide an operable connection between the two, such as by a snap-fit configuration, an insertion and twist configuration, etc. In certain configurations, the hand-held control unit has a structure configured to receive the proximal end of the elongated member.

In some instances, the hand-held control unit may be re-used simply by wiping down the hand-held control unit following a given procedure and then attaching a new elongated member to the hand-held control unit. In other instances, to provide for desired sterility to the hand-held control unit, the device may include a removable sterile covering attached to the proximal end of the elongated member that is configured to seal the hand-held control unit from the environment. This sterile covering (e.g., in the form of a sheath as described in greater detail below) may be a disposable sterile handle cover that uses a flexible bag, a portion of which is affixed to and sealed to the proximal end of the disposable elongated member. Where desired, the sterile covering may include an integrated clear monitor cover, which may be rigid and configured to conform to the monitor screen. In some instances, the cover may be configured to provide for touch screen interaction with the monitor. As indicated above, the hand-held control unit may include a manual controller. In such instances, the sterile covering may include a flexible rubber boot for mechanical controller sealing, i.e., a boot portion configured to associated with the manual controller. In addition, the sterile covering may include a seal at a region associated with the proximal end of the hand-held control unit. In these instances, the open side of sterile cover prior to use may be conveniently located at the proximal end. Following positioning of the cover around the hand-held control unit, the open side may be mechanically attached to the handle and closed by a validated sealing method. The sterile cover of these embodiments is configured such that when employed, it does not inhibit handle controls or elongated structure and monitor actuation.

As stated before, in some instances, the hand-held control unit may be a hand-held medical device (such as, for example, a tissue visualization device as described in U.S. application Ser. No. 12/501,336, the disclosure of which is hereby incorporated by reference) that has been configured to further include an RF energy source.

### Adapter

In some aspects of the invention, an adapter is provided that includes the RF energy source. For example, in some embodiments, the adapter includes an electrical energy source, a charge accumulator, voltage converter, and a RF signal generator, wherein the voltage converter, charge accumulator, and RF signal generator operably couple the electrical energy source to a plasma generator on an elongated member (e.g., RF probe). Furthermore, in some instances, the adapter may additionally include a bandpass filter and/or RF tuner.

In some aspects of the invention, the adapter is configured to operably and removably couple to a hand-held minimally dimensioned medical device. In some embodiments, the adapter may be configured to removably couple to a minimally dimensioned visualization device (such as, for example, a tissue visualization device as described in U.S. application Ser. No. 12/501,336, the disclosure of which is hereby incorporated by reference) that has been configured to couple to the adapter. For example, the visualization device may be configured to include a removable section that removes so that the adapter may operably couple in place of the removable section. It should be understood that the adapter of the present invention may be configured to removably couple and operate with a variety of medical devices other than a visualization device.

The size of the adapter may vary depending on the particular application and design consideration (e.g., how many batteries are required, whether a transformer is included, etc.). Generally, the adapter is large enough to house the RF energy source components and yet be minimally sized to maintain the hand-held nature of the device. In some instances, the adapter is smaller than five times the size of the hand-held device, including smaller than three times the size of the device, such as smaller than two times the size of the device. For example, in some instances, the device may be smaller than the size of the hand-held device. Furthermore, the weight of the adapter may vary and depends largely on the components included within. For instance, components such as batteries and transformers may provide extra weight to the adapter. The weight of the adapter may vary, but in some instances ranges from 10 lbs or less, including 5 lbs or less, such 3 lbs or less. In some instances, the weight of the hand-held control may weigh 2 lbs or less, such as 1 lb or less, including 0.5 lbs or less.

As the adapter is removably coupled to a hand-held medical device, it is configured to maintain the hand-held nature of the device—e.g., remain amenable to gripping by the human adult hand. The adapter may vary in shape and is generally shaped to couple to the hand-held device without inhibiting or negatively affecting the use of the device by the user—e.g., inhibiting movement of the device, inhibiting field of vision for the user, etc. In some instances, the adapter is configured to removably couple to the hand-held device in a manner such that it is positioned below the hand-held device when coupled. For example, the adapter may be arc-shaped or u-shaped and positioned below the hand-held device so as to provide a space between the inner arc or “u” of the adapter and the hand-held device, thus allowing the user to grip the hand-held device without the adapter obstructing the grip. In another example, the adapter is rectangularly shaped and positioned below the hand-held device when coupled—e.g., extending lengthwise downward from the device. In such case, the adapter may couple to the proximal or distal end of the device and still allow the user to grip the device. In some instances, the adapter may be configured to allow the user to grip the adapter when coupled to the device—e.g., forming a gun-shape with the device. Additionally, the adapter may be configured to include switches or other control elements, such as an activation switch to activate RF energy.

The adapter may be coupled to the hand-held device using a variety of mechanisms—e.g., hinge, magnet, Velcro, ball and socket, etc. Furthermore, the adapter may couple to the hand-held device at one or more interface locations. For example, if the adapter is arc-shaped or u-shaped, the adapter may couple to the device at one end of the arc-shaped housing, or at both ends of the arc-shaped housing, etc. Electrical contacts may be included at the interface locations (both on the adapter and on the hand-held device) to form an electrical path between the adapter and the hand-held device. The electrical path may be used to provide control signals from the hand-held device to the adapter. For example, the activation of RF energy may be initiated by an activation element on the hand-held device and control signal provided via the electrical path to activate RF energy generation and delivery. In instances where the RF probe further includes visualization sensors, switches and control elements on the hand-held device may still be used to provide and control the visualization capabilities and the RF energy capabilities.

### Additional Components And Functionality

In some aspects of the invention, the RF tissue modulation devices are configured to include additional components and the associated functionalities of the additional components. For example, the elongated member may further include components such as visualization sensors, lumens, illuminators, etc.

In some embodiments, the RF tissue modulation devices further include a visualization sensor integrated at the distal end of the elongated member, such that the visualization sensor is integrated with the elongated member. As the visualization sensor is integrated with the elongated member, it cannot be removed from the remainder of the elongated member without significantly compromising the structure and functionality of the elongated member. Accordingly, the devices of the present invention are distinguished from devices which include a “working channel” through which a separate autonomous device is passed through. In contrast to such devices, since the visualization sensor of the present device is integrated with the elongated member, it is not a separate device from the elongated member that is merely present in a working channel of the elongated member and which can be removed from the working channel of such an elongated member without structurally compromising the elongated member in any way. The visualization sensor may be integrated with the elongated member by a variety of different configurations. Integrated configurations include configurations where the visualization sensor is fixed relative to the distal end of the elongated member, as well as configurations where the visualization sensor is movable to some extent relative to the distal end of the elongated member. Movement of the visualization sensor may also be provided relative to the distal end of the elongated member, but then fixed with respect to another component present at the distal end, such as the plasma generator, a distal end integrated illuminator, etc. Specific configurations of interest are further described below in connection with the figures.

Visualization sensors of interest include miniature imaging sensors that have a cross sectional area which is sufficiently small for its intended use and yet retains a sufficiently high matrix resolution. Imaging sensors of interest are those that include a photosensitive component, e.g., array of photosensitive elements that convert light into electrons, coupled to a circuitry component, such as an integrated circuit. The integrated circuit may be configured to obtain and integrate the signals from the photosensitive array and output image data, which image data may in turn be conveyed to an extra-corporeal device configured to receive the data and display it to a user. The image sensors of these embodiments may be viewed as integrated circuit image sensors. The integrated circuit component of these sensors may include a variety of different types of functionalities, including but not limited to: image signal processing, memory, and data transmission circuitry to transmit data from the visualization sensor to an extra-corporeal location, etc. The miniature imaging sensors may be present in a module which further includes one or more of a housing, a lens component made up of one or more lenses positioned relative to the photosensitive component so as to focus images on the photosensitive component, one or more filters, polarized members, etc. Specific types of miniature imaging sensors of interest include complementary metal-oxide-semiconductor (CMOS) sensors and charge-coupled device (CCD) sensors. The sensors may have any convenient configuration, including circular, square, rectangular, etc. Visualization sensors of interest may have a longest cross sectional dimension that varies depending on the particular embodiment, where in some instances the longest cross sectional dimension (e.g., diameter) is 4.0 mm or less, such as 3.5 mm or less, including 3.0 mm or less, such as 2.5 mm or less, including 2.0 mm or less, including 1.5 mm or less, including 1.0 mm or less. Within a given imaging module, the sensor component may be located some distances from the lens or lenses of the module, where this distance may vary, such as 10 mm or less, including 7 mm or less, e.g., 6 mm or less.

Imaging sensors of interest may be either frontside or backside illumination sensors, and have sufficiently small dimensions while maintaining sufficient functionality to be integrated at the distal end of the elongated members of the devices of the invention. Aspects of these sensors are further described in one or more the following U.S. patents, the disclosures of which are herein incorporated by reference: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

As summarized above, the visualization sensor is located at the distal end of the elongated member, such that the visualization sensor is a distal end visualization sensor. In these instances, the visualization sensor is located at or near the distal end of the elongated member. Accordingly, it is positioned at 3 mm or closer to the distal end, such as at 2 mm or closer to the distal end, including at 1 mm or closer to the distal end. In some instances, the visualization sensor is located at the distal end of the elongated member. The visualization sensor may provide for front viewing and/or side viewing, as desired. Accordingly, the visualization sensor may be configured to provide image data as seen in the forward direction from the distal end of the elongated member. Alternatively, the visualization sensor may be configured to provide image data as seen from the side of the elongate member. In yet other embodiments, a visualization sensor may be configured to provide image data from both the front and the side, e.g., where the image sensor faces at an angle that is less than 90° relative to the longitudinal axis of the elongated member.

Components of the visualization sensor, e.g., the integrated circuit, one or more lenses, etc., may be present in a housing. The housing may have any convenient configuration, where the particular configuration may be chosen based on location of the sensor, direction of view of the sensor, etc. The housing may be fabricated from any convenient material. In some instances, non-conductive materials, e.g., polymeric materials, are employed.

Visualization sensors may further include functionality for conveying image data to an extra-corporeal device, such as an image display device, of a system. In some instances, a wired connection, e.g., in the form of a signal cable (or other type of signal conveyance element), may be present to connect the visualization sensor at the distal end to a device at the proximal end of the elongate member, e.g., in the form of one or more wires running along the length of the elongate member from the distal to the proximal end. In some instances, the visualization sensor is coupled to a conductive member (e.g., cable or analogous structure) that conductively connects the visualization sensor to a proximal end location of the elongated member. Alternatively, wireless communication protocols may be employed, e.g., where the visualization sensor is operably coupled to a wireless data transmitter, which may be positioned at the distal end of the elongated member (including integrated into the visualization sensor, at some position along the elongated member or at the proximal end of the device, e.g., at a location of the proximal end of the elongated member or associated with the handle of the device).

In some instances, the distal end integrated visualization sensor is present as an RF-shielded visualization module. As the visualization sensor module of these embodiments is RF-shielded, the visualization sensor module includes an RF shield that substantially inhibits, if not completely prevents, an ambient RF field from reaching and interacting with circuitry of the visualization sensor. As such, the RF shield is a structure which substantially inhibits, if not completely prevents, ambient RF energy (e.g., as provided by a distal end RF electrode, as described in greater detail blow) from impacting the circuitry function of the visualization sensor.

Visualization sensor modules of devices of the invention include at least a visualization sensor. In certain embodiments, the devices may further include a conductive member that conductively connects the visualization sensor with another location of the device, such as a proximal end location. Additional components may also be present in the visualization sensor module, where these components are described in greater detail below.

The RF shield of the visualization sensor module may have a variety of different configurations. The RF shield may include an enclosure element or elements which serve to shield the circuitry of the visualization sensor from an ambient RF field. In some instances, the RF shield is a grounded conductive enclosure component or components which are associated with the visualization sensor, conductive member and other components of the visualization sensor module. In some instances, the visualization sensor of the visualization sensor module is present in a housing, where the housing may include a grounded outer conductive layer which serves as an RF shield component. In these instances, the RF shield is an outer grounded conductive layer. The conductive enclosure or enclosures of the RF-shielded visualization sensor module may be fabricated from a variety of different conductive materials, such as metals, metal alloys, etc., where specific conductive materials of interest include, but are not limited to: copper foils and the like. In certain instances, the RF shield is a metallic layer. This layer, when present, may vary in thickness, but in some instances has a thickness ranging from 0.2 mm to 0.7 mm, such as 0.3 mm to 0.6 mm and including 0.4 mm to 0.5 mm. Additional details regarding RF-shielded visualization modules may be found in U.S. application Ser. No. 12/437,865; the disclosure of which is herein incorporated by reference.

Where desired, the devices may include one or more illumination elements configured to illuminate a target tissue location so that the location can be modified by the plasma generator and/or visualized with a visualization sensor, e.g., as described above. A variety of different types of light sources may be employed as illumination elements (also referred to herein as illuminators), so long as their dimensions are such that they can be positioned at the distal end of the elongated member. The light sources may be integrated with a given component (e.g., elongated member) such that they are configured relative to the component such that the light source element cannot be removed from the remainder of the component without significantly compromising the structure of the component. As such, the integrated illuminators of these embodiments are not readily removable from the remainder of the component, such that the illuminator and remainder of the component form an inter-related whole. The light sources may be light emitting diodes (LEDs) configured to emit light of the desired wavelength range, or optical conveyance elements, e.g., optical fibers, configured to convey light of the desired wavelength range from a location other than the distal end of the elongate member, e.g., a location at the proximal end of the elongate member, to the distal end of the elongate member. The physical location of the light source, e.g., LED, may vary, such as any location in the elongated member, in the hand-held control unit, etc.

As with the image sensors, the light sources may include a conductive element, e.g., wire, or an optical fiber, which runs the length of the elongated member to provide for power and control of the light sources from a location outside the body, e.g., an extracorporeal control device.

Where desired, the light sources may include a diffusion element to provide for uniform illumination of the target tissue site. Any convenient diffusion element may be employed, including but not limited to a translucent cover or layer (fabricated from any convenient translucent material) through which light from the light source passes and is thus diffused. In those embodiments of the invention where the system includes two or more illumination elements, the illumination elements may emit light of the same wavelength or they may be spectrally distinct light sources, where by “spectrally distinct” is meant that the light sources emit light at wavelengths that do not substantially overlap, such as white light and infra-red light. In certain embodiments, an illumination configuration as described in U.S. application Ser. Nos. 12/269,770 and 12/269,772 (the disclosures of which are herein incorporated by reference) is present in the device.

Distal end integrated illuminators may have any convenient configuration. Configurations of interest have various cross sectional shapes, including but not limited to circular, ovoid, rectangular (including square), irregular, etc. In some instances the configuration of the integrated illuminator is configured to conform with the configuration of the integrated visualization sensor such that the cross sectional area of the two components is maximized within the overall minimal cross sectional area available at the distal end of the elongated member. For example, the configurations of the integrated visualization sensor and illuminators may be such that the integrated visualization sensor may occupy a first portion of the available cross sectional area of the distal end of the elongated member (such as 40% or more, including 50% or 60% or more of the total available cross sectional area of the distal end of the elongated member) and the integrated illuminator may occupy a substantial portion of the remainder of the cross sectional area, such as 60% or more, 70% or more, or 80% or more of the remainder of the cross sectional area.

In one configuration of interest, the integrated illuminator has a crescent configuration. The crescent configuration may have dimensions configured to confirm with walls of the elongated member and a circular visualization sensor. In another configuration of interest, the integrated illuminator has an annular configuration, e.g., where conforms to the inner walls of the elongated member or makes up the walls of the elongated member, e.g., as described in greater detail below. This configuration may be of interest where the visualization sensor is positioned at the center of the distal end of the elongated member.

In some instances, the elongated member comprises an annular wall configured to conduct light to the elongated member distal end from a proximal end source. The distal end of this annular wall may be viewed as an integrated illuminator, as described above. In these instances, the walls of the elongated structure which collective make up the annular wall are fabricated from a translucent material which conducts light from a source apart from the distal end, e.g., from the proximal end, to the distal end. Where desired, a reflective coating may be provided on the outside of the translucent elongated member to internally reflect light provided from a remote source, e.g., such as an LED at the proximal end, to the distal end of the device. Any convenient reflective coating material may be employed.

Also of interest are integrated illuminators that include a fluid filled structure that is configured to conduct light to the elongated member distal end from a proximal end source. Such a structure may be a lumen that extends along a length of the elongated structure from a proximal end light source to the distal end of the elongated structure. When present, such lumens may have a longest cross section that varies, ranging in some instances from 0.5 to 4.0 mm, such as 0.5 to 3.5 mm, including 0.5 to 3.0 mm. The lumens may have any convenient cross sectional shape, including but not limited to circular, square, rectangular, triangular, semi-circular, trapezoidal, irregular, etc., as desired. The fluid filled structure may be filled with any convenient translucent fluid, where fluids of interest include aqueous fluids, e.g., water, saline, etc., organic fluids, such as heavy mineral oil (e.g., mineral oil having a specific gravity greater than or equal to about 0.86 and preferably between about 0.86 and 0.905), and the like.

As indicated above, certain instances of the integrated illuminators are made up of an elongated member integrated light conveyance structure, e.g., optical fiber, light conductive annular wall, light conducting fluid filled structure, etc., which is coupled to a proximal end light source. In some instances, the proximal end light source is a forward focused LED. Of interest are in such embodiments are bright LEDs, e.g., LEDs having a brightness of 100 mcd or more, such as 300 mcd or more, and in some instances 500 mcd or more, 1000 mcd or more, 1500 mcd or more. In some instances, the brightness ranges from 100 to 2000 mcd, such as 300 to 1500 mcd. The LED may be coupled with a forward focusing lens that is, in turn, coupled to the light conveyance structure.

In some instances, the proximal end LED may be coupled to the light conveyance structure in a manner such that substantially all, if not all, light emitted by the LED is input into the light conveyance structure. Alternatively, the LED and focusing lens may be configured such that at least a portion of the light emitted by the LED is directed along the outer surface of the elongated member. In these instances, the forward focused light emitting diode is configured to direct light along the outer surface of the elongated member. As such, light from the proximal end LED travels along the outer surface of the elongated member to the distal end of the elongated member.

In some instances, the RF tissue modulation devices of the invention are configured to reduce coupling of light directly from the integrated illuminator to the visualization sensor. In other words, the devices are structures so that substantially all, if not all, of the light emitted by the integrated illuminator at the distal end of the elongated structure is prevented from directly reaching the visualization sensor. In this manner, the majority, if not all, of the light that reaches the visualization sensor is reflected light, which reflected light is converted to image data by the visualization sensor. In order to substantially prevent, if not inhibit, light from the integrated illuminator from directly reaching the integrated visualization sensor, the device may include a distal end polarized member. By distal end polarized member is meant a structure or combination of structures that have been polarized in some manner sufficient to achieve the desired purpose of reducing, if not eliminating, light from the integrated illuminator directly reaching the integrated visualization sensor. In some embodiments, the light from an LED is polarized by a first polarizer (linearly or circularly) as it enters at lens or prism at the distal tip of the elongated member. A visualization sensor, such as CMOS sensor, also has a polarizer directly in front of it, with this second polarizer being complimentary to the first polarizer so that any light reflected by the outer prism surface into the visualization sensor will be blocked by this polarizer. Light passing through the first polarizer and reflected by the surrounding tissue will have random polarization, so roughly half of this light will pass through the second polarizer to reach the visualization sensor and be converted to image data. The distal end polarized member may be a cover lens, e.g., for forward viewing elongated members, or a prism, e.g., for off-axis viewing elongated members, such as described in greater detail below.

In some instances, the distal end of the elongated member includes an off-axis visualization module that is configured so that the visualization sensor obtains data from a field of view that is not parallel to the longitudinal axis of the elongated member. With an off-axis visualization module, the field of view of the visualization sensor is at an angle relative to the longitudinal axis of the elongated member, where this angle may range in some instances from 5 to 90°, such as 45 to 75°, e.g., 30°. The off-axis visualization module may include any convenient light guide which collects light from an off-axis field of view and conveys the collected light to the visualization sensor. In some instances, the off-axis visualization module is a prism.

Depending on the particular device embodiment, the elongated member may or may not include one or more lumens that extend at least partially along its length. When present, the lumens may vary in diameter and may be employed for a variety of different purposes, such as irrigation, aspiration, electrical isolation (for example of conductive members, such as wires), as a mechanical guide, etc., as reviewed in greater detail below. When present, such lumens may have a longest cross section that varies, ranging in some instances from 0.5 to 5.0 mm, such as 1.0 to 4.5 mm, including 1.0 to 4.0 mm. The lumens may have any convenient cross sectional shape, including but not limited to circular, square, rectangular, triangular, semi-circular, trapezoidal, irregular, etc., as desired. These lumens may be provided for a variety of different functions, including as conveyance structures for providing access of devices, compositions, etc. to the distal end of the elongated member, as described in greater detail below. Such lumens may be employed as a “working channel”.

In some embodiments, an integrated articulation mechanism that imparts steerability to at least the distal end of the elongated member or a component thereof is also present in the device, such that the elongated member is the elongated member is configured for distal end articulation. By “steerability” is meant the ability to maneuver or orient the distal end of the elongated member or component thereof as desired during a procedure, e.g., by using controls positioned at the proximal end of the device, e.g., on the hand-held control unit. In these embodiments, the devices include a steerability mechanism (or one or more elements located at the distal end of the elongated member) which renders the desired elongated member distal end or component thereof maneuverable as desired through proximal end control. As such, the term “steerability”, as used herein, refers to a mechanism that provides a user steering functionality, such as the ability to change direction in a desired manner, such as by moving left, right, up or down relative to the initial direction. The steering functionality can be provided by a variety of different mechanisms. Examples of suitable mechanisms include, but are not limited to one or more wires, tubes, plates, meshes or combinations thereof, made from appropriate materials, such as shape memory materials, music wire, etc.

In some instances, the distal end of the elongated member is provided with a distinct, additional capability that allows it to be independently rotated about its longitudinal axis when a significant portion of the operating handle is maintained in a fixed position, as discussed in greater detail below. The extent of distal component articulations of the invention may vary, such as from −180 to +180°; e.g., −90 to +90°. Alternatively, the distal probe tip articulations may range from 0 to 360°, such as 0 to +180°, and including 0 to +90°, with provisions for rotating the entire probe about its axis so that the full range of angles is accessible on either side of the axis of the probe, e.g., as described in greater detail below. Rotation of the elongated member may be accomplished via any convenient approach, e.g., through the use of motors, such as described in greater detail below. Articulation mechanisms of interest are further described in published PCT Application Publication Nos. WO 2009029639; WO 2008/094444; WO 2008/094439 and WO 2008/094436; the disclosures of which are herein incorporated by reference. Specific articulation configurations of interest are further described in connection with the figures, below, as well as in U.S. application Ser. No. 12/422,176; the disclosure of which is herein incorporated by reference.

In addition to the distal end integrated visualization sensor, e.g., as described in greater detail above, devices of the invention may include a distal end integrated non-visualization sensor. In other words, the devices may include one or more non-visualization sensors that are integrated at the distal end of the elongated member. The one or more non-visualization sensors are sensors that are configured to obtain non-visual data from a target location. Non-visual data of interest includes, but is not limited to: temperature, pressure, pH, elasticity, impedance, conductivity, distance, size, etc. Non-visualization sensors of interest include those configured to obtain one or more types of the non-visual data of interest. Examples of sensors that may be integrated at the distal end include, but are not limited to: temperature sensors, pressure sensors, pH sensors, impedance sensors, conductivity sensors, elasticity sensors, etc. Specific types of sensors include, but are not limited to: thermistors, strain gauges, membrane containing sensors, MEMS sensors, electrodes, light sensors, etc. The choice of a specific type of sensor will depend on the nature of the non-visual data of interest. For example, a pressure sensor can detect the force applied to a target tissue as it is deformed to determine the elastic modulus of the target tissue. A temperature sensor can be employed to detect locally elevated temperatures (which can be used to differentiate different types of tissue, such as to different normal and tumor tissue (where tumors exhibit increased bloodflow and therefore a higher temperature)). A properly collimated laser beam could be used to determine the distance to objects in the device field of view or the length scale of objects in the device field of view. When present, the integrated non-visualization sensor or sensors may be configured to complement other distal end components of the devices, so as to minimize any impact on the outer dimension of the distal end, e.g., in ways analogous to those described above in connection with integrated illumination elements.

In some instances, the RF tissue modulation devices further include a second tissue modifier other than the plasma generator. Tissue modifiers are components that interact with tissue in some manner to modify the tissue in a desired way. The term modify is used broadly to refer to changing in some way, including cutting the tissue, ablating the tissue, delivering an agent(s) to the tissue, freezing the tissue, etc. As such, of interest as tissue modifiers are tissue cutters, tissue ablators, tissue freezing/heating elements, agent delivery devices, etc. Tissue cutters of interest include, but are not limited to: blades, liquid jet devices, lasers and the like. Tissue ablators of interest include, but are not limited to ablation devices, such as devices for delivery ultrasonic energy (e.g., as employed in ultrasonic ablation), devices for delivering plasma energy, devices for delivering radiofrequency (RF) energy, devices for delivering microwave energy, etc. Energy transfer devices of interest include, but are not limited to: devices for modulating the temperature of tissue, e.g., freezing or heating devices, etc. In some embodiments, the tissue modifier is not a tissue modifier that achieves tissue modification by clamping, clasping or grasping of tissue such as may be accomplished by devices that trap tissue between opposing surfaces (e.g., jaw-like devices). In these embodiments, the tissue modification device is not an element that is configured to apply mechanical force to tear tissue, e.g., by trapping tissue between opposing surfaces.

In some instances, the tissue modifier is a low-profile tissue modifier, such as a low-profile biopsy tool or a low-profile cutter. Such low-profile tissue modifiers are include tissue cutting structure positioned at the distal of the elongated member. Because the biopsy or cutting tool is low-profile, its presence at the distal end of the elongated member does not substantially increase the outer diameter of the elongated member. In some instances, the presence of the low-profile biopsy tool increase the outer diameter of the elongated member by 2 mm or less, such as 1.5 mm or less, including 1 mm or less. The configuration of the low-profile biopsy tool may vary. In some instances, the low-profile biopsy tool comprises an annular cutting member concentrically disposed about the distal end of the elongated member and configured to be moved relative to the distal end of the elongated member in a manner sufficient to engage tissue. The annular cutting member may or may not be configured as a complete ring structure, where the ring structure is movable in a longitudinal manner relative to the distal end of the elongated member (such that it may be moved along the elongated member towards and away from the proximal end of the elongated member). The distal edge of the ring structure may be movable some distance beyond the distal end of elongated member, where this distance may vary and in some instances is 10 mm or less, such as 5 mm or less, including 3 mm or less. The distal edge of the ring structure may be sharp in order to penetrate tissue, and may include one or more tissue retaining structures, such as barbs, hooks, lips, etc., which are configured to engage the tissue and stably associate the engaged tissue with the ring structure, e.g., when the ring structure is moved longitudinally along the elongated member towards the proximal end. Also of interest are cutting tools, e.g., as described

In some instances, the RF tissue modulation devices may include a collimated laser configured to emit collimated laser light from a distal region of the elongated member, such as the distal end of the elongated member. The collimated laser components of these embodiments may be configured for use for a variety of purposes, such as but not limited to: anatomical feature identification, anatomical feature assessment of sizes and distances within the field of view of the visualization sensor, etc.

In some instances, the devices may include a stereoscopic image module. By stereoscopic image module is meant a functional module that provides a stereoscopic image from image data obtained by the device. As such, the module provides a user via the monitor with the perception of a three-dimensional view of an image produced from the image data obtained by the device. The module is described in terms of “images”, and it should be understood that the description applies equally to still images and video. Further details regarding stereoscopic image modules and image recognition modules can be found in U.S. application Ser. Nos. 12/501,336 and 12/269,770; the disclosures of which are herein incorporated by reference.

In certain embodiments, devices of the invention include an image recognition module. Image recognition modules of interest are those that are configured to receive image data and compare the received image data with a reference that includes at least one of color descriptor data and anatomical descriptor data to make a determination as to whether an alert signal should be generated. Further details regarding image recognition modules are provided in U.S. application Ser. Nos. 12/501,336 and 12/437,186; the disclosures of which are herein incorporated by reference.

In some embodiments, the devices may include a conveyance structure configured to convey an item between the distal end of the elongated member and an entry port positioned at a proximal end of the device, e.g., associated with the proximal end of the elongated member or associated with the hand-held control unit. This conveyance structure may have any convenient configuration, where in some instances it is a “working channel” disposed within the elongated member. When present as a working channel, the channel may have an outer diameter that varies, and in some instances has an outer diameter of 3 mm or less, such as 2 mm or less and including 1 mm or less. The conveyance structure may be configured to transport items, e.g., fluids, medicines, devices, to an internal target site or from an internal target site. As such, the proximal end entry port of the conveyance structure may vary, and may be configured to be operably coupled to a variety of different types of components, such as but not limited to: aspiration units, fluid reservoirs, device actuators, etc.

As indicated elsewhere, devices of the invention may be configured for wireless data transmission, e.g., to provide for one or more of: transmission of data between various component of the device, transmission of data between components of the device and another device, such as hospital information system, separate monitor, etc. Any convenient wireless communication protocol may be employed, where in some instances wireless communication is implemented as one or more wireless communication modules.

A video processor module may be present and be configured to control the one or more distinct visualization sensors by sending camera control data to a camera module including the visualization sensor(s). The video processor may also be configured to receive sensor data from one ore more sensors and/or tools; and further, may be configured to control the sensors and/or tools by sending sensor control data to a sensor module including the one or more sensors and/or tools. The various sensors may include, but are not limited to, sensors relating to pressure, temperature, elasticity, ultrasound acoustic impedance, laser pointer to identify and/or measure difference to sensors, etc. The various tools may include, but are not limited to, a measurement scale, teardrop probe, biopsy probe, forceps, scissors, implant device, IR lighting, ultrasound measurement device, cutting tool, etc. Depending on the specific application and sensor/tool implemented, sensor data may also be included with the image data for processing by the stereoscopic image module, in order to provide the stereographic images.

In certain instances, the devices of the invention include an updatable control module, by which is meant that the devices are configured so that one or more control algorithms of the device may be updated. Updating may be achieved using any convenient protocol, such as transmitting updated algorithm data to the control module using a wire connection (e.g., via a USB port on the device) or a wireless communication protocol. The content of the update may vary. In some instances, a hand-held control unit is updated to configure the unit to be used with a particular elongated member. In this fashion, the same hand-held control units may be employed with two or more different elongated members that may differ by function and have different components. In some instances, the update information may be transmitted from the particular elongated member itself, such that upon operable connection of the elongated member to the hand-held control unit, update information is transferred from the elongated member to the hand-held control unit that updates the control module of the hand-held control unit such that it can operate with that particular elongated member. The update information may also include general functional updates, such that the hand-held control unit can be updated at any desired time to include one or more additional software features and/or modify one or more existing programs of the device. The update information can be provided from any source, e.g., a particular elongated member, the internet, etc.

The devices of the invention may be fabricated using any convenient materials or combination thereof, including but not limited to: metallic materials such as tungsten, stainless steel alloys, platinum or its alloys, titanium or its alloys, molybdenum or its alloys, and nickel or its alloys, etc; polymeric materials, such as polytetrafluoroethylene, polyimide, PEEK, and the like; ceramics, such as alumina (e.g., STEATITE™ alumina, MAECOR™ alumina), etc.

### Illustrated Embodiments

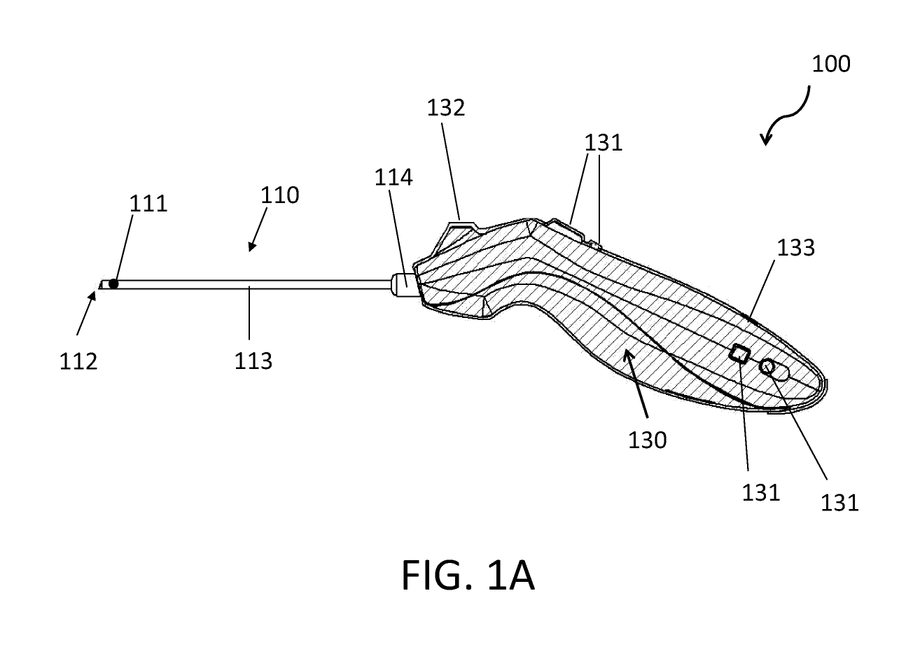

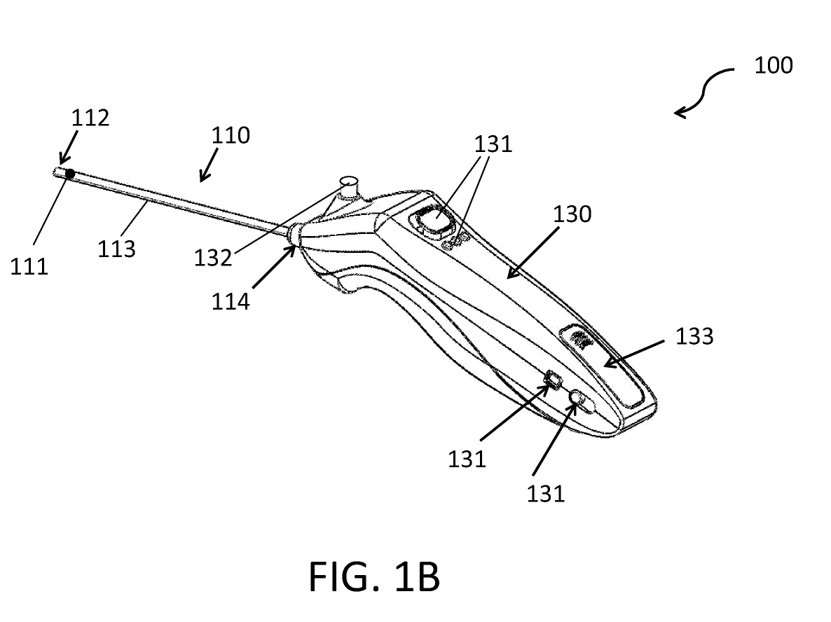

Turning now to the figures, FIGS. 1A-1B illustrate a side view and perspective view, respectively, of an RF tissue modification device comprising a hand-held control unit and RF probe, according to some embodiments. Both figures are described together in the following paragraphs.

The RF tissue modulation device 100 is shown including a hand-held control unit 130 and a removably coupled elongated member 110 having a plasma generator 111 at the distal end (also referred to herein as a “RF probe 110”). From an external view, the RF probe 110, as shown, includes a distal end tip 112, and tubular structure 113, and a mechanical connector 114 to removably couple to the hand-held control unit 130. The hand-held control unit 130, from an external view may include various control switches 131 for controlling the device—e.g., activating delivery of RF energy to the plasma generator 111, turning power off and on, controlling the rotation or articulation of the RF probe 110, controlling functions associated with illuminators, visualization, etc., if such capabilities are present, etc. It should be understood that the term switch is used generally and may include any various types of control elements, such as keys, buttons, wheels, etc. Furthermore, it should be understood that the control switches 131 may be positioned in various locations on the hand-held control unit 130.

While not required, positioning control switches 131 in locations on the hand-held control unit 130 that can be accessed by the user while gripping the control unit 130 provides the advantage of being more user-friendly. This may be especially advantageous for control switches 131 expected to be used more frequently. For example, one of the control switches 131 may control the delivery of RF energy. Another one of the control switches 131 may, for example, control motor rotation and three positions available for controlling the motor rotation, one position to rotate the motor clockwise, one position to rotate the motor counterclockwise, and a position in the center that is neutral.

Furthermore, as shown in FIG. 1A, there may be a battery door 133 for the purpose of accessing the electrical energy source inside. As stated above, the electrical energy source may include one or more DC batteries, for example. The DC batteries may be rechargeable or non-rechargeable batteries. In some embodiments, the hand-held control unit may be configured to removably couple to a docking station, cradle, plug, etc. (not shown) to recharge the electrical energy source.

Internally, the hand-held control unit 130 includes RF energy source components as described above. The hand-held control unit 130 may include, for example, the electrical energy source, a voltage converter, charge accumulator, and RF signal generator (not shown). Example embodiments of the RF energy source are described in further detail in later figures illustrating example block diagrams of the RF energy source. It should be understood that additional circuitry such as wiring, LEDs, control units (e.g., microcontrollers and/or microprocessors), memory units (e.g., volatile and non-volatile memory) may also be included within the hand-held control unit.

An RF line (not shown) is positioned along the RF probe to electrically couple the hand-held control unit 130 and the plasma generator 111 positioned at the distal end of the RF probe 110. The RF line may be, for example, conductive wiring extending within the RF probe 110 from the mechanical connector 114 to the RF electrode (not shown) of the plasma generator 111. In some instances, RF probe 110 includes RF shielding as described above.

In some instances, the RF probe 110 may include additional components other than the plasma generator 111 (e.g., visualization sensors, illumination elements, lumens, etc.). For example, in some embodiments, a visualization sensor may be included at a distal end of the RF probe 110, and a monitor coupled to the hand-held control unit 130 at an optional monitor connector 132. In some embodiments, the hand-held control unit 130 includes a built in monitor or display.

Hand-held minimally dimensioned diagnostic devices having integrated distal end visualization sensors and other additional components are discussed in U.S. application Ser. No. 12/501,336, the disclosure of which is hereby incorporated by reference. The components, their configurations, and operations thereof, described within the disclosure may also apply here to the RF probe 110 and hand-held control unit 130 of the RF tissue modulation devices 100, when such components are present. For example, when visualization capabilities are included within device 100, hand-held control unit 130 may include associated circuitry such as an image processor, video processor, and/or stereoscopic image module, as described in U.S. application Ser. No. 12/501,336. Additionally, tissue modification devices having tissue modifiers and other additional components are discussed in Provisional Application Ser. No. 61/082,774, U.S. application Ser. No. 12/422,176, and International patent application Ser. No. 09/51446, the disclosures of which are herein incorporated by reference. The components, their configurations, and operations thereof, described within these disclosures may also apply here to the RF probe 110 and hand-held control unit 130 of the RF tissue modulation devices 100, when such components are present.

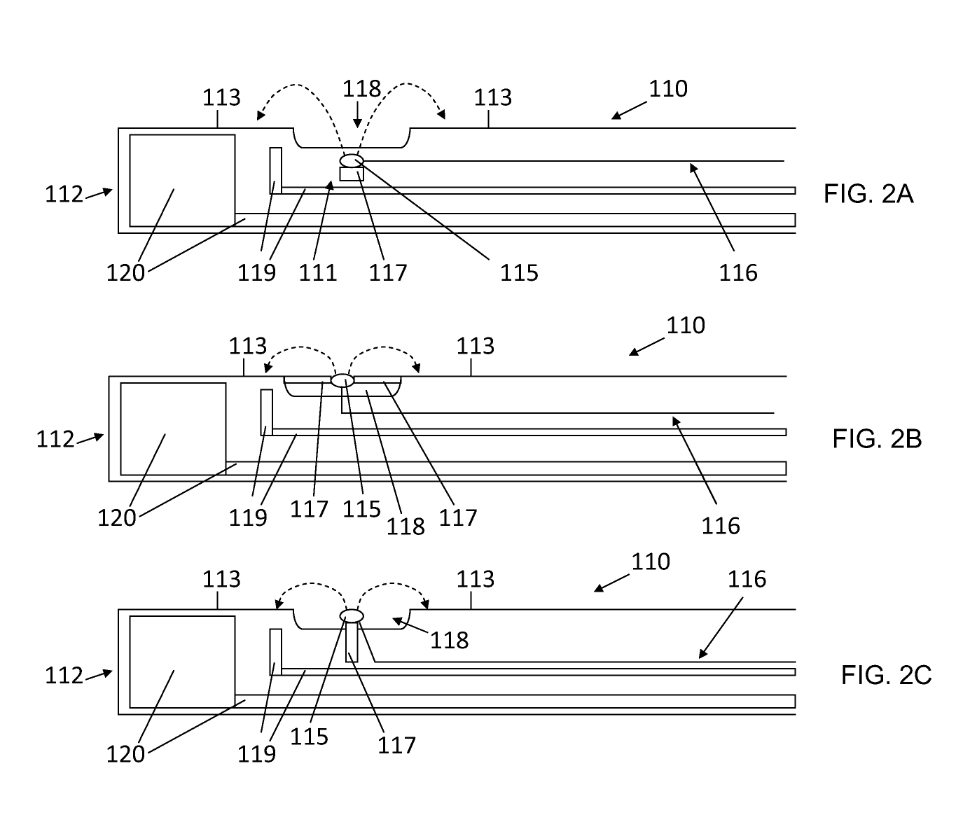

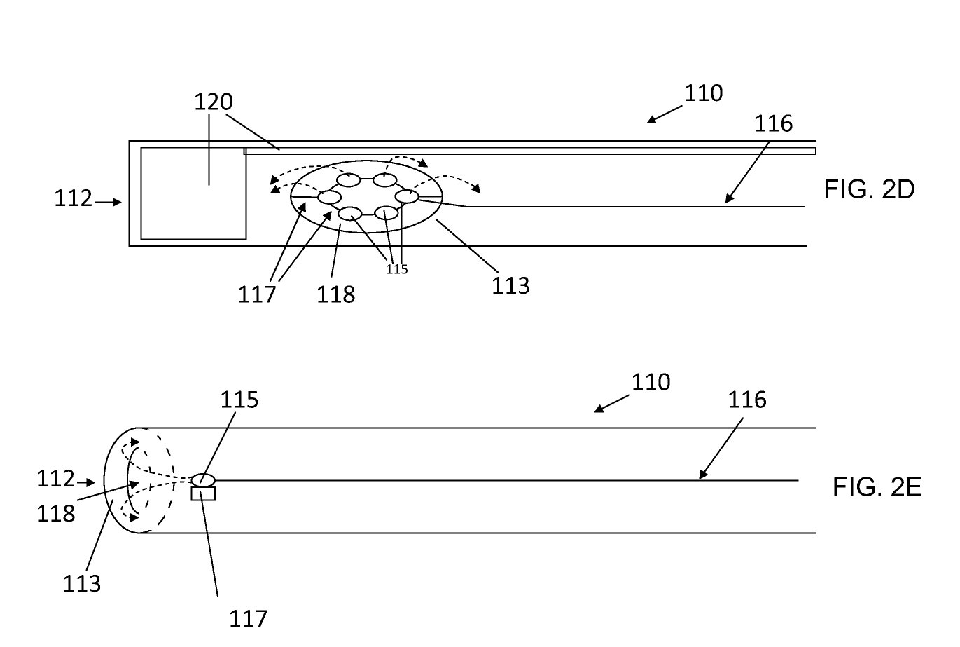

FIGS. 2A-2E illustrate a distal end of an elongated member 110 including a plasma generator 111, according to some embodiments. Plasma generator 111 is shown to include a conductive member 115 functioning as an RF electrode. The plasma generator may also include insulators and/or other conductive members such as other electrodes. Conductive member 115 is maintained in position by insulator 117. The conductive member 115 is coupled to RF line 116. RF line 116 is shown extending from the conductive member 115 at the distal end of the elongated member 110 down the length of the elongated member to the proximal end of the elongated member 110. RF line 116 provides an electrical connection between the RF energy source (not shown) and the conductive member 115 such that RF energy (e.g., high voltage modulated RF signals as described above) may be delivered to conductive member 115 from RF energy source when RF energy is activated. When RF energy is activated and received by plasma generator 111, plasma generator 111 produces a plasma between the conductive member 115 and outer surface 113, for example, as represented by the dotted arrows illustrated in FIGS. 2A-2E.

For FIGS. 2A-2D, elongated member 110 is shown to further include additional components 120 (such as earlier described visualization sensors, illuminator elements, etc.) also at the distal end of the elongated member 110. Additional components may also include components running the length of the elongated member 110—e.g., wires, fiber optics, etc. It should be understood that the position of the additional components may vary depending on application, and are represented generally in FIGS. 2A-2D.

As further shown in FIGS. 2A-2D, elongated member may also include an RF shield 119 within the elongated member 110 and adjacent to the RF line 116 and/or RF electrode 115. RF shield 119 provides an ambient RF barrier between the additional components 120 and RF line 116 and/or conductive member 115.

FIG. 2A illustrates a cross sectional side view of an elongated member 110, according to one embodiment. Elongated member 110 includes an outer surface 113, distal end opening 118 within the outer surface 113, and distal end tip 112. In this embodiment, distal end opening 118 is positioned over conductive member 115. When RF energy is delivered to plasma generator 111 via RF line 116, a plasma is generated between the conductive member 115 and outer surface of the elongated member 113, as represented by the dotted arrows.

FIG. 2B illustrates a cross sectional side view of an elongated member 110, according to one embodiment. Elongated member 110 includes an outer surface 113, distal end opening 118 within the outer surface 113, and distal end tip 112. Conductive member 115 is positioned within the distal end opening 118 by insulator 117. In this embodiment, insulator 117 is shown extending from elongated member 110 near the perimeter of the opening 118. When RF energy is delivered to plasma generator 111 via RF line 116, a plasma is generated between the conductive member 115 and outer surface 113, as represented by the dotted arrows.

FIG. 2C illustrates a cross sectional side view of an elongated member 110, according to one embodiment. Elongated member 110 includes an outer surface 113, distal end opening 118 within the outer surface 113, and distal end tip 112. Conductive member 115 is positioned within the distal end opening 118 by insulator 117. In this embodiment, insulator 117 extends from within the elongated member 110. When RF energy is delivered to plasma generator 111 via RF line 116, a plasma is generated between the conductive member 115 and outer surface 113, as represented by the dotted arrows.

FIG. 2D illustrates a cross sectional top view of an elongated member 110, according to one embodiment. Elongated member 110 includes an outer surface 113, distal end opening 118 within the outer surface 113, and distal end tip 112. Conductive member 115 is positioned within the distal end opening 118 by insulator 117. In this embodiment, insulator 117 is shown extending from elongated member 110 near the perimeter of the opening 118. Furthermore, in this embodiment, multiple insulators 117 and conductive members 115 are shown. As shown, insulators 117 may be positioned between conductive members 115 to maintain the conductive members 115 in position. It should be understood that RF line may extend within the insulator 117 in some instances—e.g., metal wiring extending through a piece of ceramic and contacting the RF electrode. When RF energy is delivered to plasma generator 111 via RF line 116, a plasma is generated between the conductive members 115 and outer surface 113, as represented by the dotted arrows.

FIG. 2E illustrates a cross sectional side view of an elongated member 110, according to one embodiment. Elongated member 110 includes an outer surface 113, distal end opening 118 within the outer surface 113, and distal end tip 112. In this embodiment, distal end opening 118 is positioned over conductive member 115 at the distal end tip 120 of the elongated member 110. When RF energy is delivered to plasma generator 111 via RF line 116, a plasma is generated between the conductive member 115 and outer surface 113, as represented by the dotted arrows. It should be understood, that while the FIG. 2E is shown not to include additional components 120, this embodiment is exemplary and additional components 120 may be included in other embodiments having the distal end opening 118 at the distal end tip 112. Moreover, it should be understood that elongated members shown in FIGS. 2A-2D, may not include additional components 120 and/or RF shielding 119 in other embodiments. It should also be understood that the embodiments shown for FIGS. 2A-2E are illustrative and are functionally represented to facilitate understanding of the configurations and placements of the components. For example, the embodiments shown are not drawn to scale and do not embody the exact shapes of the components used.

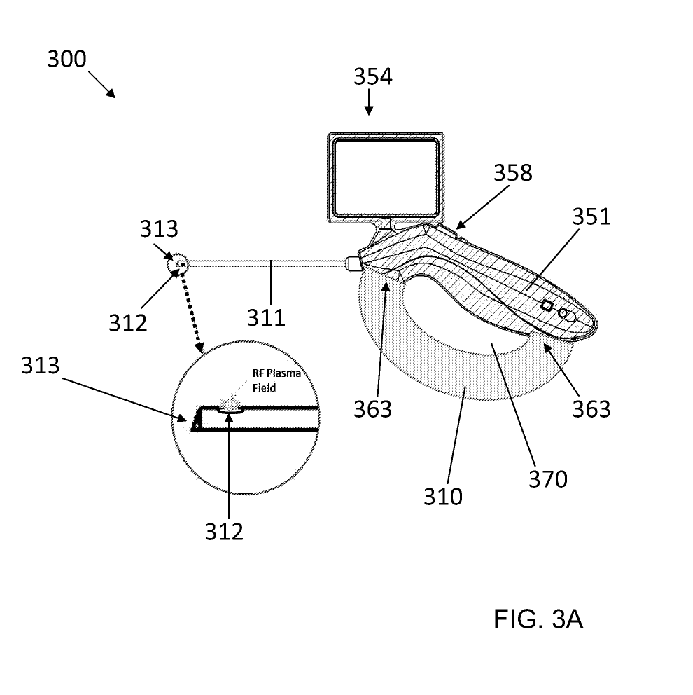

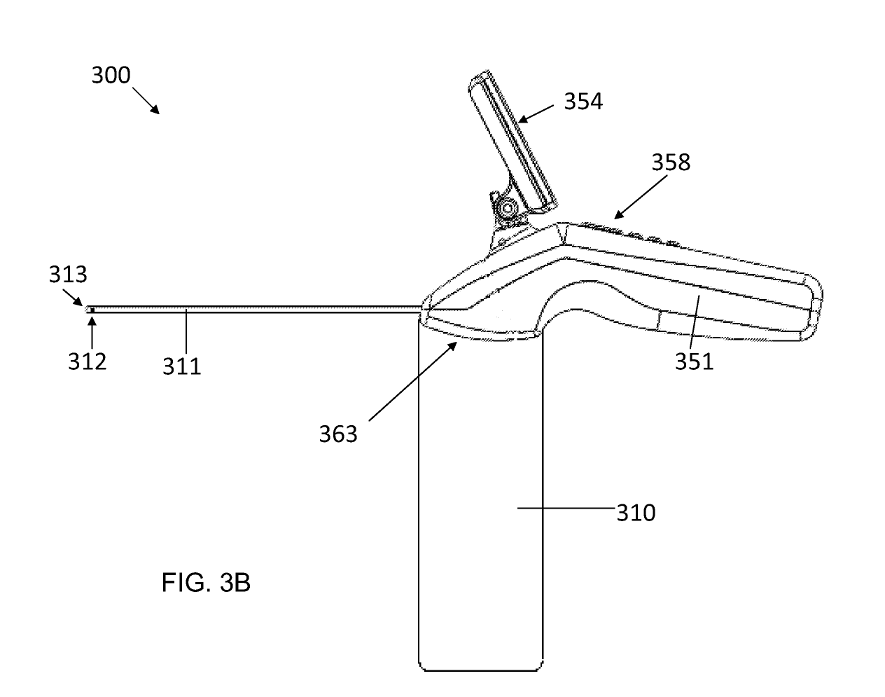

FIG. 3A-3B illustrate RF tissue modulation devices including an adapter, elongated member, and hand-held piece, according to some embodiments. It should be understood that in some instances the hand-held piece and elongated member may function without the adapter as a diagnostic device, such as a visualization device. For example, the visualization device may be similar to the visualization devices described in U.S. application Ser. No. 12/501,336, except configured to removably couple to the adapter.

As shown in FIG. 3A, RF tissue modulation device 300 includes a hand-held piece 351 and an elongated member 311 (e.g., a RF probe) coupled to the hand-held piece 351. Hand-held piece 351 is shown having a monitor 354 and control switches 358 coupled to hand-held piece 351. In this embodiment, elongated member 311 is removably coupled to the hand-held piece 351 at a distal end of the hand-held piece 351. The elongated member 352 includes a RF generator 312 and visualization sensor 313 at a distal end of the elongated member 311 used to provide visualization to monitor 354 coupled to the hand-held piece 351. The distal end of the elongated member 311 is shown close up in FIG. 3, as represented by the dotted arrow and circled sections. Furthermore, as explained earlier, additional components as well as the visualization sensor may be included in the RF probe 311—e.g., illuminators, lumens, etc.

While in this example embodiment, a visualization sensor 313 is included in the elongated member 311, it should be understood that a visualization sensor may not be included in another embodiment. Additionally, it should be understood that the elongated member 311 may be removed from hand-held piece 351 and a new elongated member may be coupled in its place. For example, in some instances, an RF probe without visualization capabilities may be coupled to the hand-held piece 351 instead. Furthermore, it should be understood that, in some instances, an elongated member (e.g., a visualization probe) without a RF plasma generator may be used in place of the RF probe, in which case the hand-held piece 351 and visualization probe function as a visualization device (with or without the adapter 310 coupled).

Adapter 310 is shown having an arc-shape or u-shape and removably coupled to hand-held piece 351. Adapter 310 is form fitted to couple to the hand-held piece 351 at interface locations 363 and to provide a space 370 between the inner arc or “u” of the adapter 310 and the hand-held piece 351, thus allowing the user to grip the hand-held piece 351 without the adapter 310 obstructing the user's grip.

Internally, adapter 310 includes RF energy source components (not shown). For example, in some embodiments, the adapter 310 includes an electrical energy source, a charge accumulator, voltage converter, and RF signal generator, wherein the voltage converter, charge accumulator, and RF signal generator operably couple the electrical energy source to the plasma generator 312 on the elongated member 311 removably and operably coupled to the hand-held piece 351. Example embodiments of the RF energy source are described in further detail in later figures illustrating example block diagrams of the RF energy source. It should be understood that additional circuitry such as wiring, LEDs, control units (e.g., microcontrollers and/or microprocessors), memory units (e.g., volatile and non-volatile memory) may also be included within the adapter. Furthermore, in some instances, the adapter 310 may additionally include a bandpass filter and/or RF tuner.

An RF line (not shown) is positioned within RF probe 311 to electrically couple the adapter 310 and the plasma generator 312 positioned at the distal end of the RF probe 311. The RF line may be, for example, conductive wiring extending within the RF probe 311 from an RF electrode (not shown) of the plasma generator 312. In some instances, RF probe 311 includes RF shielding as described above.

Adapter 310 is configured to couple to the hand-held piece 351 at interface locations 363. Electrical contacts (not shown) may be provided at interface locations 363 on both the adapter 310 and the hand-held piece 351 to provide an electrical path between the two. The electrical path provides an electrical path for the delivery of RF energy from the adapter to the plasma generator. Furthermore, the electrical path provides a communication path between the adapter 310 and the hand-held piece 351

As stated above, the RF probe 311 coupled to the adapter 310 includes a visualization sensor 313 in addition to a plasma generator 312. In such case, the hand-held piece 351 and adapter 310 are configured such that the hand-held piece 351 may operate with the visualization sensors 313 and plasma generator 312 on the RF probe 311. Further, the hand-held piece 351 includes various switches 358 to control functions of the hand-held piece 351 and adapter 310—e.g., switches to activate the delivery of RF energy to the plasma generator, switches for controlling visualization, lighting, rotation, articulation, etc. When RF energy is activated (e.g., by the user depressing a corresponding control switch 358, the RF energy source within adapter generates RF energy (e.g., the high voltage modulated RF signal described earlier) and delivers it to the plasma generator 312 via the RF line.

Adapter 310 may further include a battery door (not shown) for removing the electrical energy source—e.g., chargeable or non-chargeable DC batteries. In some instances, the rechargeable batteries cannot be removed by the user and the adapter configured to removably couple to a docking station, cradle, plug, etc. In such case, the adapter may include a corresponding charging plug, port, etc. In some instances, the adapter is configured to be charged via electrical contacts at the interface locations 363.

While this embodiment is described as having two interface locations, it should be understood that in other embodiments, the RF tissue modulation device may include another number of interface locations—e.g., one. Furthermore, it should be understood that when there are more than one interface location, electrical contacts may be included at one or more of the interface locations. Moreover, the electrical path for delivery of RF energy is not required to be at the same interface location of the electrical path for communication between the hand-held piece and the adapter.

The description above for FIG. 3A applies to FIG. 3B as well, except in FIG. 3B the adapter 310 is generally shaped as a rectangle as opposed to an arc or u-shape, and is configured to couple to the hand-held piece 351. The rectangular shaped adapter 310 is configured to removably and operably couple to interface location 363 of hand-held piece 351 of RF tissue modulation device 300. Interface location 363 may, for example, include a socket, plug, or other coupling mechanism for coupling the adapter 310 to the hand-held piece 351. Upon coupling, contacts (not shown) from the adapter 310 at interface location 363 and contacts (not shown) from the hand-held piece form an electrical path for delivery of control signals, as well as delivery of RF energy from the adapter 310 to the plasma generator 312, as similarly described above. It should be understood that a variety of shapes and interface locations may be implemented without compromising the underlying principles of the invention.

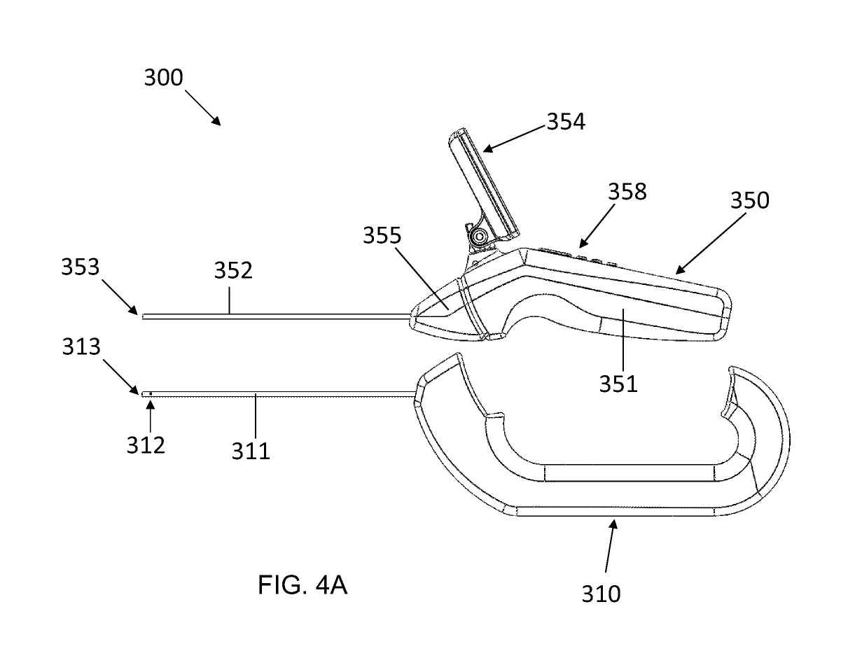

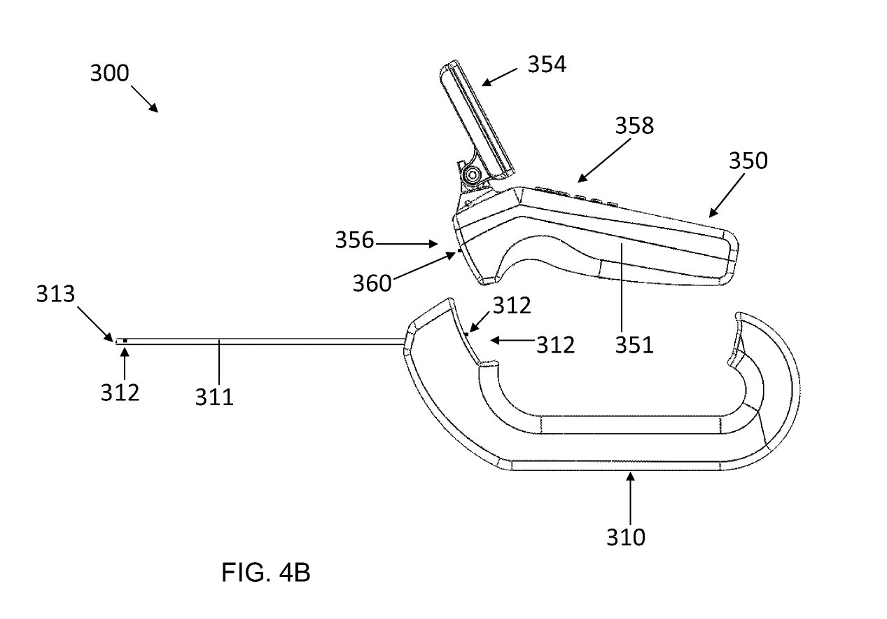

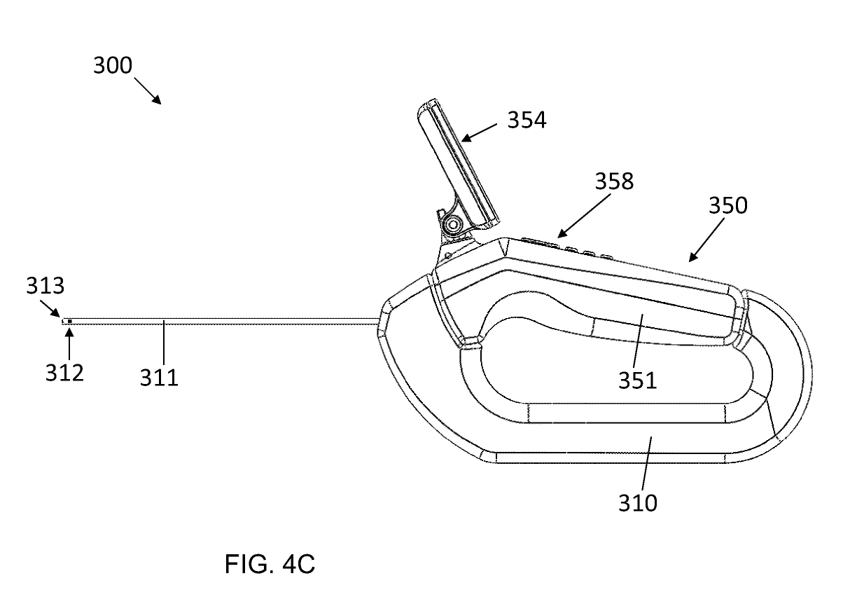

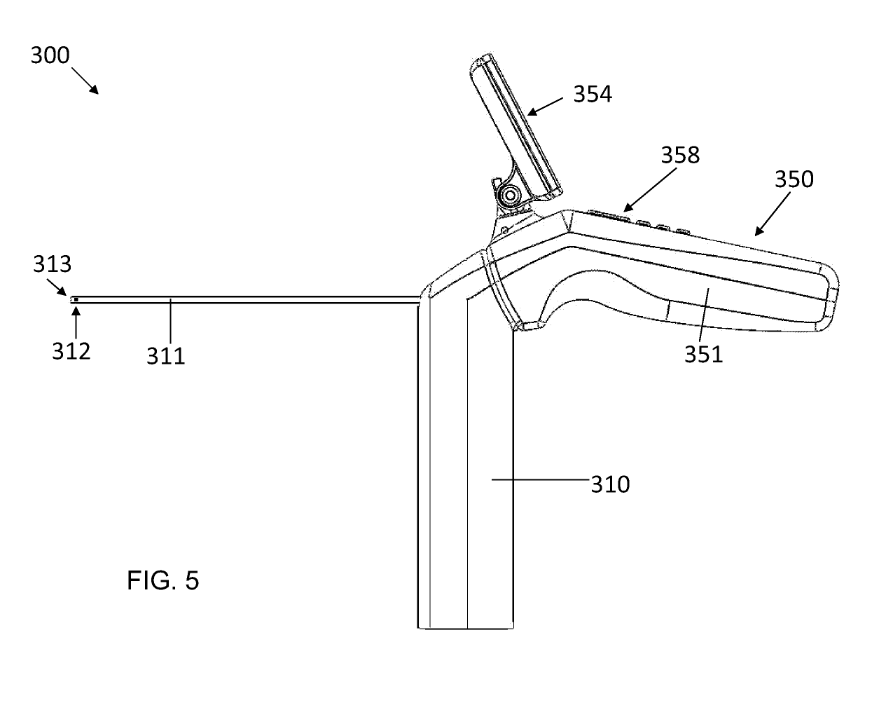

FIGS. 4A-4C and FIG. 5 illustrate an RF tissue modulation device 300 including an adapter 310 and diagnostic device 350 (also referred to herein as “visualization device”), according to one embodiment. The visualization device may be similar to the visualization devices described in U.S. application Ser. No. 12/501,336, except configured to removably couple to the adapter. More specifically, FIGS. 4A-4C and FIG. 5 illustrate various embodiments where the elongated member (e.g., RF probe) is removably coupled to the adapter.

As shown in FIG. 4A, visualization device 350 includes a hand-held piece 351 and an elongated member 352 (e.g., a visualization probe) coupled to the hand-held piece 351. Hand-held piece is shown having a monitor 354 and control switches 358 coupled to hand-held piece 351. The elongated member 352 includes a visualization sensor 353 at a distal end of the elongated member 352 used to provide visualization to monitor 354 coupled to the hand-held piece 351. In this embodiment, elongated member 352 is removably coupled to the hand-held piece 351 at a removable section 355 of the hand-held piece 351. Removable section 355 is removed when adapter 310 is to be operably coupled to the hand-held piece 351.

Adapter 310 is shown having an arc-shape or u-shape with RF probe 311 removably coupled to adapter 310. The RF probe includes a plasma generator 312 and visualization sensor 313 at a distal end. Adapter 310 is form fitted to couple to the visualization device 350 at interface location 356 and to provide a space between the inner arc or “u” of the adapter 310 and the hand-held device 350, thus allowing the user to grip the hand-held device 350 without the adapter 310 obstructing the user's grip.

Internally, adapter includes RF energy source components (not shown). For example, in some embodiments, the adapter includes an electrical energy source, a charge accumulator, voltage converter, and RF signal generator, wherein the voltage converter, charge accumulator, and RF signal generator operably couple the electrical energy source to the plasma generator 312 on an elongated member 311 (e.g., RF probe) removably and operably coupled to the adapter 310. Example embodiments of the RF energy source are described in further detail in later figures illustrating example block diagrams of the RF energy source. It should be understood that additional circuitry such as wiring, LEDs, control units (e.g., microcontrollers and/or microprocessors), memory units (e.g., volatile and non-volatile memory) may also be included within the adapter. Furthermore, in some instances, the adapter 310 may additionally include a bandpass filter and/or RF tuner.

Elongated member 311 is shown removably coupled to adapter 310 and includes a plasma generator 312 and visualization sensor 313 at a distal end of the elongated member 311. While in this example embodiment, a visualization sensor is included in the elongated member, it should be understood that at visualization sensor may not be included in another embodiment. Furthermore, as explained earlier, additional components as well as the visualization sensor may be included in the RF probe 311—e.g., illuminators, lumens, etc.

An RF line (not shown) is positioned within RF probe 311 to electrically couple the adapter 310 and the plasma generator 312 positioned at the distal end of the RF probe 311. The RF line may be, for example, conductive wiring extending within the RF probe 311 from an RF electrode (not shown) of the plasma generator 312. In some instances, RF probe 311 includes RF shielding as described above.

To couple the adapter 310 to the visualization device 350, the removable section 355 of the hand-held piece 351, along with elongated member 352, are removed, as illustrated in FIG. 4B. Removable section 355 is removably coupled to hand-held piece 351 at an interface location 356. Interface location 356 may include electrical contacts 360 that contact contacts on the removable section 355, thus forming an electrical path between the hand-held piece 351 and visualization probe 352.

Adapter 310 is configured to couple to the hand-held piece 351 at an interface location 356 where the removable section 355 was coupled, as illustrated in FIG. 4C. In this way, the electrical contacts 360 at interface location 357 on the hand-held piece 351 that were providing an electrical path between the hand-held piece 351 and the elongated member 352 are now used to provide an electrical path between the hand-held piece 351 and contacts 361 on the interface location 357 of adapter 310 when coupled.

As stated above, the RF probe 311 coupled to the adapter 310 includes a visualization sensor 313 in addition to a plasma generator 312. In such case, the hand-held device 350 and adapter 310 are configured such that the hand-held device 350 may operate with the visualization sensors 313 and plasma generator 312 on the RF probe 311. Further, the visualization device 350 includes various switches 358 to control functions of the device 350 and adapter 310—e.g., switches to activate the delivery of RF energy to the plasma generator, switched for controlling visualization, lighting, rotation, articulation, etc. When RF energy is activated (e.g., by the user depressing a corresponding control switch 358, the RF energy source within adapter generates RF energy (e.g., the high voltage modulated RF signal described earlier) and delivers it to the plasma generator 312 via the RF line.

While in this example embodiment, removable section is removed in order to operably couple the hand-held piece, in another embodiment, the adapter operably couples to the hand-held piece without requiring a removable section to be included on the hand-held piece. In such case, the RF probe 311 removably couples to the hand-held piece at the same location that the visualization probe 352 is removably coupled.

Adapter 310 may further include a battery door (not shown) for removing the electrical energy source—e.g., chargeable or non-chargeable DC batteries. In some instances, the rechargeable batteries cannot be removed by the user and the adapter configured to removably couple to a docking station, cradle, plug, etc. In such case, the adapter may include a corresponding charging plug, port, etc. In some instances, the adapter is configured to be charged via electrical contacts 361.

FIG. 5 illustrates an RF tissue modulation device 300 including an adapter 310 and diagnostic device 350 (also referred to herein as “visualization device”), according to one embodiment. The description above for FIGS. 4A-4C apply to FIG. 5 as well, except in FIG. 5 the adapter 310 is generally shaped as a rectangle as opposed to an arc or u-shape. The rectangular shaped adapter 310 is configured to removably and operably couple to the interface location 356 of hand-held piece 351 of visualization device 350.

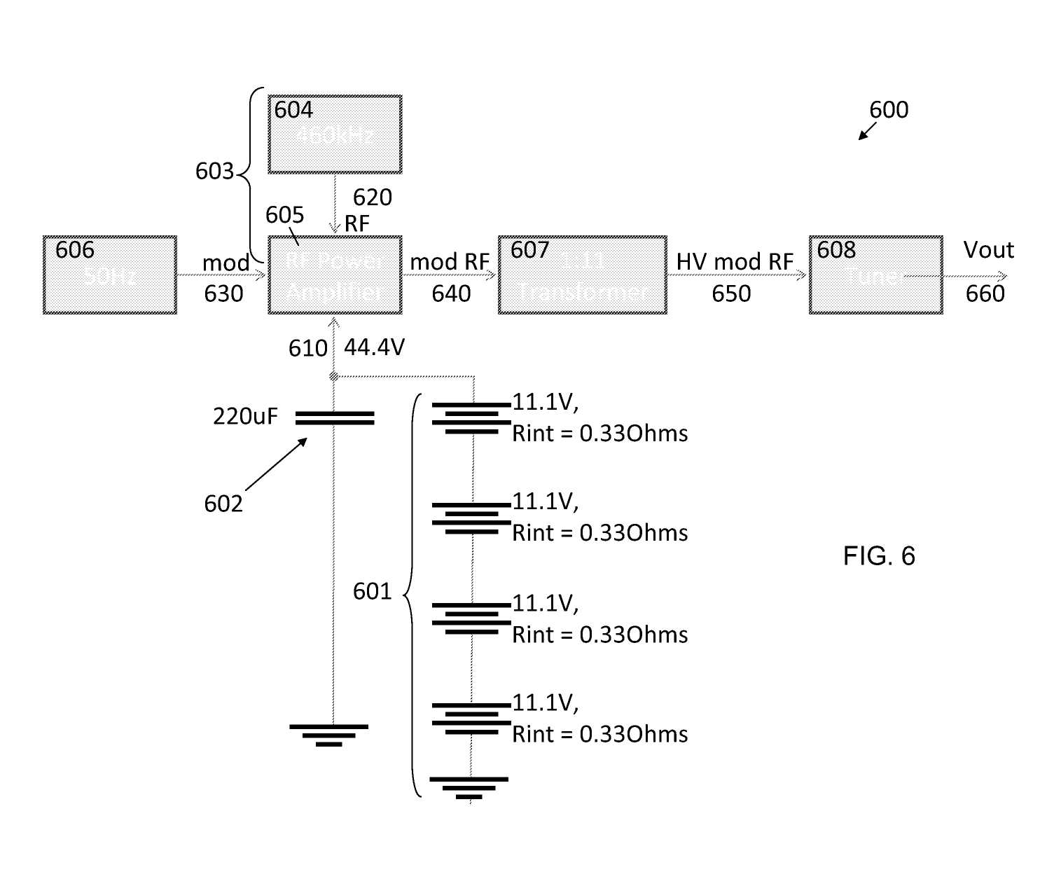

Turning now to the RF energy source, FIG. 6 illustrates a functional block diagram of an RF energy source, according to one embodiment. As shown, RF energy source 600 includes an electrical energy source 601 coupled to a charge accumulator 602. Electrical energy source 601 provides electrical energy for storage in charge accumulator 602. Electrical energy source 601 may comprise one or more DC power sources (e.g., batteries) to provide the electrical energy for storage in charge accumulator 602, shown here as a capacitor. For example, in the embodiment shown, electrical energy source 601 comprises four 11.1 volt batteries connected in series and provides a combined DC voltage of 44.4 volts across charge accumulator 602. Charge accumulator 602 and electrical energy source 601 are shown coupled to RF signal generator 603.

In the embodiment shown, DC voltage from electrical energy source 601 is provided across charge accumulator 602 and charge is stored therein. In some instances, charging may occur when delivery of RF energy to the plasma generator is not activated by the user. When RF energy is activated, charging of the charge accumulator 602 is interrupted and the stored energy in the charge accumulator 602 is discharged. For example, electrical energy source 601 may be disconnected from charge accumulator 602 by a switch (not shown) triggered by a control signal received from the hand-held control unit or medical device upon depression of the control switch for activation of RF energy. For example, charge accumulator 602 may be decoupled from electrical energy source 601 and begin discharging. The discharging of the charge accumulator 602 provides a voltage signal 610 to the RF signal generator 603.

The RF tissue modulation devices may be configured to deliver RF energy from the RF energy source to the plasma generator for a therapeutic duration. The therapeutic duration may range, for example, from minutes or less, such as 1 minute or less, including 30 seconds or less, such as 10 seconds or less. In some instances, the therapeutic duration may range from 1 to 2 seconds. The therapeutic duration may be controlled using a variety of implementations. For example, in some instances, a timer (not shown) may be used to return switches back to positions for charging—e.g., switches that couple/uncouple the charge accumulator to the electrical energy source. As another example, in some instances, recharging of the charge accumulator may not occur until the user releases the activation switch—e.g., thus coupling the charge accumulator back to the electrical energy source.

After delivery of RF energy to the plasma generator, the electrical energy source 601 is again coupled to the charge accumulator 602 and charging may occur again. In some instances, the RF tissue modulation device is configured to recharge the charge accumulator within a minimum recharge period between plasma generation. The minimum recharge period may range, for example, from 10 minutes or less, including 5 minutes or less, such as 3 minutes or less. In some instances, the minimum recharge period ranges from 1 to 2 minutes. Various recharge periods can be implemented by varying, for example, battery and capacitance sizes.

RF signal generator 603 is shown comprising an RF power amplifier 605 and RF clock source 604. RF power amplifier 605 is coupled to RF clock source 604 and receives an RF clock signal 620 as its input. RF power amplifier 605 receives the RF clock signal 620, as well as a bias voltage 610 from charge accumulator 602, and generates an amplified RF signal with an operating frequency based on the RF clock signal 620 and peak voltage based on the bias voltage 610 (e.g., in this case approximately 44 volts).

RF signal generator 603 is shown also coupled to a second clock source 606 providing a second clock signal 630 for generating a modulated output signal based on the second clock signal 630. Again, the modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 50 Hz. The RF signal is modulated at the modulation frequency based on a second clock and a modulated RF signal is output from the RF signal generator 603. Thus, in such case, the RF signal generator 603 generates a modulated RF signal 640 and outputs it to voltage converter 607.

RF signal generator 603 is coupled to a voltage converter 607, such as the transformer shown. Voltage converter 607 steps up the voltage level of the modulated RF signal 640 received and outputs a high-voltage modulated RF signal 650. While it should be understood that a voltage boost is not necessarily required if the electrical energy source 601 provides sufficient voltage to begin with; however, in typical applications, practical design considerations (e.g., weight and size) limit the batteries to a voltage level which requires further boosting. In the embodiment shown, voltage converter 607 is a 1:11 transformer which boosts the voltage level of the modulated RF signal 640 to a high-voltage modulated RF signal 650 with approximately 11 times the voltage amplitude. For example, if the modulated RF signal 640 is at approximately 44 volts, then the high-voltage modulated RF signal 650 would have a voltage of approximately 484 volts.

Also shown in this embodiment is an optional RF tuner 608 coupled to voltage converter 607. RF tuner 608 receives the high voltage modulated RF signal 650 and outputs a signal 660 to the plasma generator—e.g., vian RF line. Signal 660 is a high voltage modulated RF signal that has been tuned as follows. The RF tuner 608 includes basic electrical elements (e.g., capacitors and inductors) which serve to tailor the output impedance of the RF energy system. The term “tailor” is intended here to have a broad interpretation, including affecting an electrical response that achieves maximum power delivery, affecting an electrical response that achieves constant power (or voltage) level under different loading conditions, affecting an electrical response that achieves different power (or voltage) levels under different loading conditions, etc. Furthermore, the elements of the RF tuner 608 can be chosen so that the output impedance is dynamically tailored, meaning the RF tuner 608 self-adjusts according to the load impedance encountered at the electrode tip. For instance, the elements may be selected so that the electrode has adequate voltage to develop a plasma corona when the electrode is placed in a saline solution (with saline solution grounded to return electrode), but then may self-adjust the voltage level to a lower threshold when the electrode contacts tissue (with tissue also grounded to return electrode, for example through the saline solution), thus dynamically maintaining the plasma corona at the electrode tip while minimizing the power delivered to the tissue and the thermal impact to surrounding tissue. RF tuner 608, when present, can provide a number of advantages. For example, delivering RF energy to target tissue through the distal tip of the electrode is challenging since RF energy experiences attenuation and reflection along the length of the conductive path from the RF energy system to the electrode tip, which can result in insertion loss. Inclusion of an RF tuner 608, e.g., as described above, can help to minimize and control insertion loss.

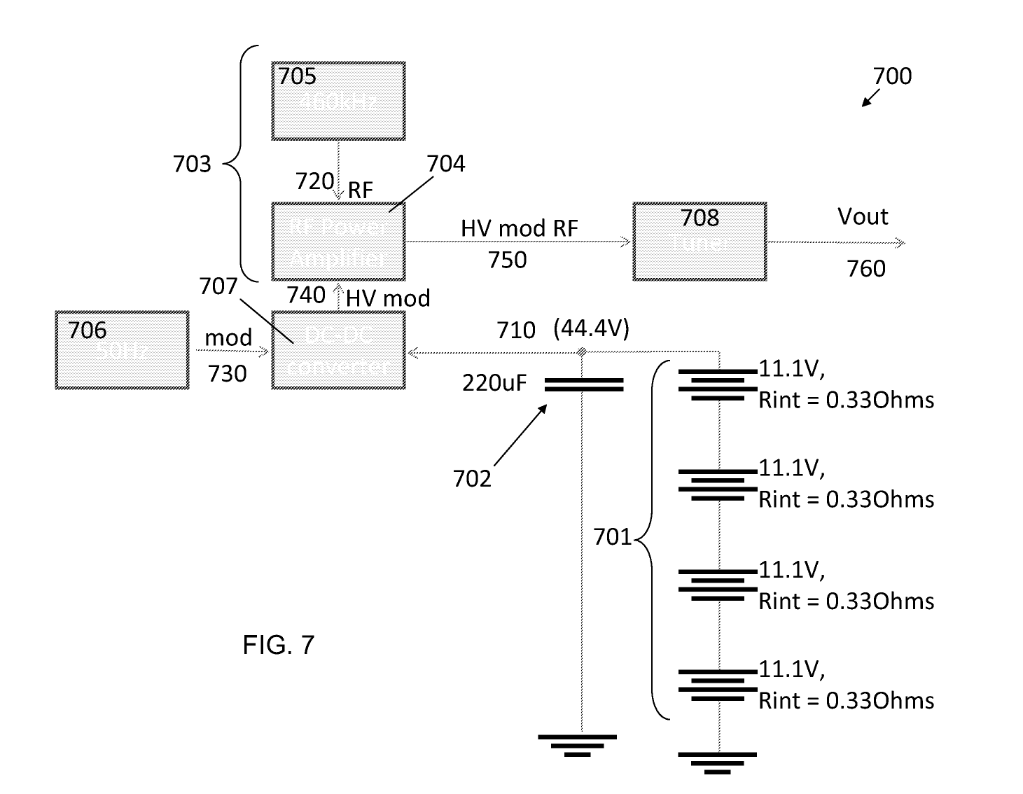

FIG. 7 illustrates a functional block diagram of an RF energy source, according to one embodiment. As shown, RF energy source 700 includes an electrical energy source 701 coupled to a charge accumulator 702. Again, electrical energy source 701 is shown as a series of 11.1 volt DC batteries to provide a voltage of approximately 44.4V to the charge accumulator 702 shown in this case to be a capacitor. Electrical energy provides the electrical energy for storage in charge accumulator 702 that discharged when activation of RF energy occurs. The above description for the charge accumulator and electrical energy source of FIG. 6 apply here as well, except the discharge of the capacitor is received by a voltage converter.

In this embodiment, voltage converter 707, shown here as a DC to DC converter, is coupled to charge accumulator 702 and receives the discharged voltage signal 710 from the charge accumulator 702. Voltage converter 707 boosts the voltage signal 710 received by the charge accumulator 702 to generate a high voltage output signal. Voltage converter 707 is also shown coupled to a clock source 706. The voltage converter 707 is configured to receive a clock signal 730 from the clock source 706 for modulation purposes and to output the high voltage signal at a modulated rate.

In some instances, the modulation at the modulation frequency comprises attenuating the amplitude of the high voltage signal based on the second clock signal. The modulation waveform (i.e., the clock signal from the clock source) may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 50 Hz.

For example, the clock signal 730 may be coupled to the enable input of voltage converter 707. In this way, the voltage converter 707 boosts the voltage signal 710 when enabled (e.g., when the clock signal 730 is high) and does not output a signal when disabled (e.g., when the clock signal 730 is low). Thus, the high voltage signal is modulated at a modulation frequency based on the clock signal from the clock source to generate a high voltage modulated signal 740.

Voltage converter 707 is also shown coupled to RF signal generator 703. RF signal generator 703 receives the high voltage modulated signal 740 from voltage converter 707 and outputs a high voltage modulated RF signal 750. RF signal generator 703 is shown comprising an RF power amplifier 704 and RF clock source 705. RF power amplifier 704 is coupled to RF clock source 705 and receives an RF clock signal 720 from the RF clock source 705. Further, RF power amplifier 704 receives the high voltage modulated signal 740 from voltage converter 707 as a bias voltage.

The RF power amplifier 704 generates an amplified RF signal with an operating frequency based on the RF clock signal 720 and peak voltage based on the bias voltage (i.e., the high voltage modulated signal 740 from voltage converter 707). The resulting high voltage modulated RF signal 750 is output by the RF signal generator 703. In some instances, as shown, the RF signal generator 703 outputs high voltage modulated RF signal 750 to an optional RF tuner 708. RF tuner 708 receives the high voltage modulated RF signal and generates a tuned high voltage modulated RF signal 760, as similarly described above for FIG. 6.

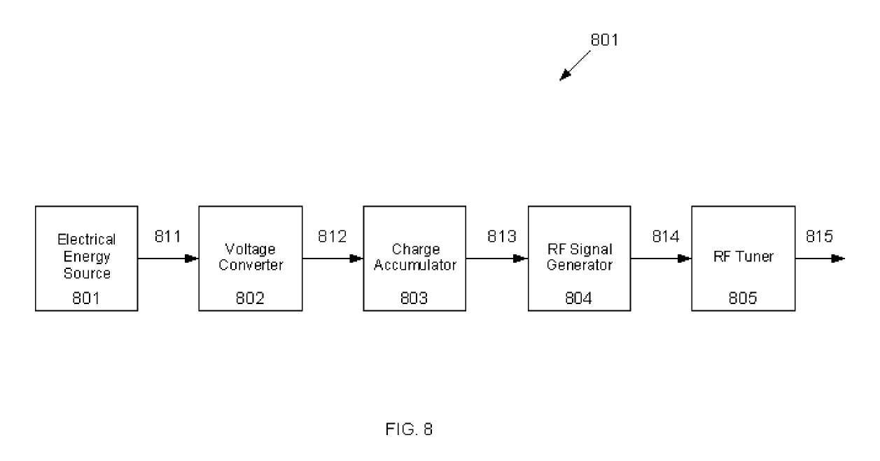

FIG. 8 illustrates a high level functional block diagram of an RF energy source 800, according to one embodiment. As shown, RF energy source 800 includes an electrical energy source 801 coupled to a voltage converter 802. The electrical energy source 801 may comprise one or more DC power sources (e.g., batteries) to provide voltage 811 to a voltage converter 802. The voltage converter 802 boosts the voltage 811 provided by the electrical energy source 801 to provide a high voltage signal 812. The voltage converter 802 is coupled to a charge accumulator 803 and the high voltage output 812 from the voltage converter 802 provides electrical energy for storage within charge accumulator 803.

Charge accumulator 803 stores the electrical energy until RF energy is activated, at which point the electrical energy is discharged from the charge accumulator 803 as a high voltage modulated output signal 813. Charge accumulator is coupled to RF signal generator 804 and high voltage modulated output signal 813 is received by RF signal generator 804. In one embodiment, charge accumulator 803 discharges the stored energy in stages. In some instances, a modulation circuit is implemented to discharge stages of energy at a specific frequency and duty cycle, thus providing the modulated aspect of the high voltage modulated output signal 813.

The RF signal generator 804 receives the high voltage modulated signal 813 from charge accumulator 803 and outputs a high voltage RF signal 814 at a specific operating frequency. The RF signal generator 804 outputs the high voltage modulated RF signal 814 to an optional RF tuner 805. RF tuner 805 receives the high voltage modulated RF signal 814 and provides a tuned high voltage modulated RF signal as similarly described above for FIG. 6.

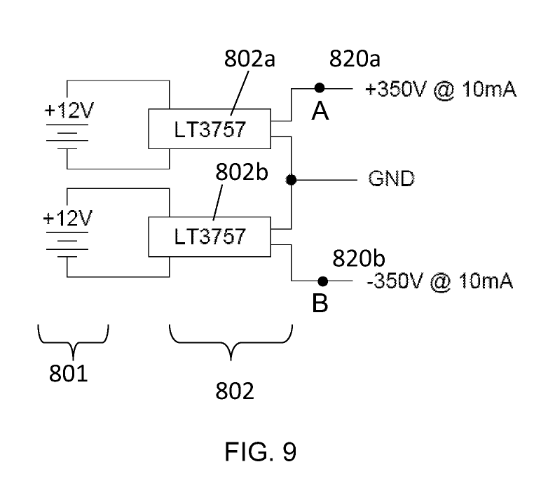

FIGS. 9-12 illustrate functional block diagrams corresponding to various elemental blocks shown in FIG. 8, according to certain embodiments. FIG. 9 illustrates a functional block diagram of the electrical energy source 801 and voltage converter 802 as shown in FIG. 8, according to one embodiment. Voltage converter 802 is shown to generally include voltage converter 802a and voltage converter 802b. In the embodiment shown, electrical energy source 801 comprises two DC power sources (e.g., batteries) with each coupled to separate voltage converters 802a,802b. The two voltage converters 802a,802b are configured to provide positive and negative high voltage rails at Point A and Point B shown in FIG. 9, respectively, with a common ground 821. A variety of devices may be used to perform such voltage conversion—e.g., two LT3757 DC-DC controllers by Linear Technologies as shown. In this embodiment shown, voltage converter 802a is configured to step up the voltage of a 12 volt battery to generate a positive high voltage output signal 820a at a positive rail. The second voltage converter 802 is configured to step up the voltage of a second 12 volt battery to generate a negative high voltage output signal 820b at a negative rail. Ranges of positive and negative high voltage outputs 820a,820b may vary depending on the particular application and design considerations. For instance, in some cases, positive and negative high voltage outputs 820a,820b may range from +/−50 volts (at e.g., approximately 1.4 mA) to +/−1000 volts (at e.g., approximately 28.5 mA), such as from +/−200 volts (at e.g., approximately 5.7 mA) to +/−500 volts (at e.g., approximately 14.2 mA), and including from +/−300 volts (at e.g., approximately 8.5 mA) to +/−400 volts (at e.g., approximately 11.4 mA). In some embodiments, the positive and negative high voltage outputs are +/−350 volts (at e.g., approximately 10 mA), as shown. Voltage levels may depend on the particular application and design considerations (e.g., voltage and current limits, etc.).

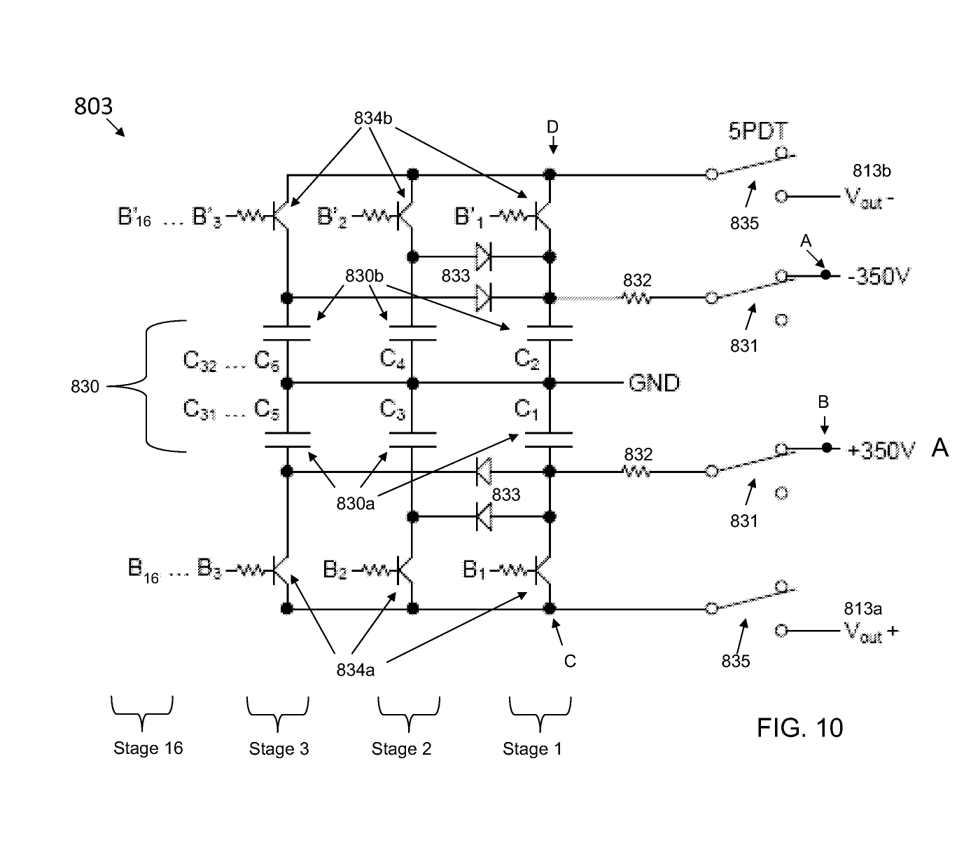

FIG. 10 illustrates a functional block diagram of the charge accumulator 803 shown in FIG. 8, according to one embodiment. Charge accumulator 803 may comprise one or more capacitors 830 configured to store electrical energy from the positive and negative high voltage outputs 820a,820b received by voltage converter 802 at corresponding Point A and Point B shown in FIG. 10. Switches 831 are shown in positions to allow the signals 820a,820b to charge the capacitors 830 when RF energy is not activated. Current associated with positive and negative voltage signals 820a,820b are provided through resistors 832 to charge capacitor 830. Diodes 833 are configured so that the capacitors are charged and discharged in stages. In other embodiments, the charge accumulator 803 is configured to charge all stages simultaneously.

In some instances, the charge accumulator 803 may be configured in stages, wherein electrical energy is stored in each stage, as represented by stages 1 through 16 shown in FIG. 10. The electrical energy can later be delivered as high voltage when RF energy is activated. For example, capacitors 830 are shown comprised of capacitor pairs—e.g., pair C1, C2; pair C3, C4, . . . pair C31, C32—with each pair referred to as being in a stage. Each pair of capacitors includes capacitor 830a associated with energy storage from the positive high voltage signal 820a received by the charge accumulator 803 at Point A, and another capacitor 830b associated with energy storage from the negative high voltage signal 820b received by the charge accumulator 803 at Point B. Point A and Point B in FIG. 10 correspond to charge accumulator 803's input lines 840 (when switch 831 is positioned accordingly), and further correspond to Point A and Point B in FIG. 9 and voltage converter 802's output lines.

Transistors 834a,834b are shown coupled to a capacitor 830a,830b, respectively. In some instances, as shown, transistors 834a,834b are bipolar junction transistors (BJT) used as switching devices. When turned on, transistor 834a is configured to provide a high voltage signal received from capacitor 830a to a positive high voltage rail at Point C. Similarly, when turned on, transistor 834b is configured to provide a negative high voltage signal received from capacitor 830b to a negative high voltage rail at Point D. It should be understood that transistors 834a,834b may also be configured to provide inverted voltage signals without compromising the underlying principles of the invention. Transistors 834a,834b are further configured to receive input signals that turn the transistor on and off. For example, transistors 834a are configured to receive signals B1-B16 at the respective base inputs of BJTs 834a to turn on the respective BJT. Similarly, transistors 834b are configured to receive signals B′ 1-B′16 at the respective base inputs of BJTs 834b to turn on the respective BJT.

Positive and negative high voltage output rails at Point C and Point D, respectively, are shown coupled to switches 835. Point C and Point D are also referred to herein as charge accumulator 803's output lines and output positive and negative high voltage signals 813a,813b, respectively, when the corresponding transistors are turned on.

The output lines of charge accumulator 803 are shown floating while the input lines of charge accumulator 803 are coupled to the output lines of voltage converter 802. Thus no RF energy provided to the plasma generator. Switches 831 and 835 are configured to switch when RF energy is activated so that charging is interrupted and accumulated charge is discharged. For example, when user activates RF energy by depressing an activation switch, for example, switches 831 for are switched such that the input lines go from the contacts coupling it to the voltage converter 802 to floating. Switches 835 for the output lines of the charge accumulator 803 are switched such that output lines go from floating to contacts coupling it to input lines of the RF signal generator 804. The switches 831 and 835 are returned to the positions shown after RF energy is delivered to the RF signal generator 804. In some instances, switches 831 and 835 are configured to switch independently. For example, after RF energy is activated, switch 835 may not switch back to the position shown in FIG. 10 until all RF energy is has been delivered to the plasma generator.

When RF energy is activated, the charge accumulator 803 is configured to discharge stored energy in each stage sequentially such that the energy from each stage is sequentially multiplexed to RF signal generator 804. The sequential rate of discharge of each stage may vary depending on desired application and design considerations. For example, each transistor pair 834a,834b in each stage may be configured to turn on when an activation voltage signal (e.g., B1-B16 and B′1-B′16) is applied to its base. In this way, an activation voltage signal may be applied to a pair of transistors 834a,834b in a first stage, and then subsequently to a pair of transistors 834a,834b in a second stage, and so on, until all stages have discharged.

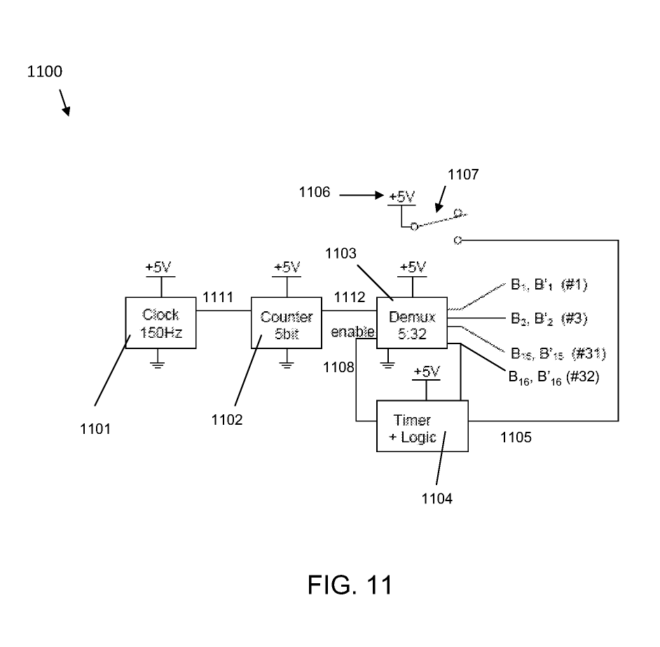

A modulation circuit (e.g., the one described in FIG. 11) may be implemented to provide the activation voltages signals sequentially to each stage at a modulated rate, as described further in FIG. 11. Thus, charge accumulator 803 receives a high voltage signal from voltage converter 802 and outputs a high voltage modulated signal on its output lines. The modulation rate can range from 1 Hz to 10 kHz, such as from 1 Hz to 500 Hz, and including from 10 Hz to 100 Hz. The duty cycle may also vary and range from 5% to 95%, such as from 25% to 75%, and including from 45% to 55%). In some embodiments, the duty cycle is approximately 50%.

FIG. 11 illustrates a functional block diagram of a modulation circuit 1100 coupled to charge accumulator 803 shown in FIG. 10, according to one embodiment. Modulation circuit 1100 is coupled to the charge accumulator 803 and outputs activation voltage signals (B1,B1′ to B16-B16′) to turn on the transistors 834a,834b in charge accumulator 803, thus discharging the stored charge in the pairs of capacitors 830a,830b at a modulated rate. More specifically, the activation voltage signals (B1, B1′ to B16-816′) from the output of the modulation circuit 110 are input into the base of the transistor and bias the transistor and turn it on and off accordingly.

In this embodiment, the modulation circuit 100 comprises a clock source 1101 (e.g., 50 Hz clock as shown) coupled to a counter 1102 (e.g., 5 bit counter as shown). Counter 1102 receives a clock signal 1111 from the clock source 1101 and provides a counting output 1112 to demultiplexer 1103—e.g., a count corresponding to each clock cycle. Demultiplexer 1103 thus receives an incremental counting signal 1112 from the counter 1102 (e.g., at 50 Hz as shown). Demultiplexer 1103 is also coupled to a timer 1104 which enables and disables the demultiplexer 1103. Timer 1104 includes a input enable line 1105 which is floating until RF energy is activated (e.g., by user depressing an activation switch) at which point the input line 1105 is connected to a power source 1106 (e.g., 5 volts as shown) via switch 1107 to enable timer circuit 1104. Switch 1107 returns to its original position thereafter (e.g., after depression of the activation switch by user. Timer 1104 provides an enable signal 1108 to the demultiplexer 1103 for a predetermined amount of time.

Demultiplexer 1103 is shown having a plurality of output lines, each coupled to respective bases of transistor pairs 834a,834b in a given stage of the charge accumulator 803. Each capacitor pair of the set is coupled to the demultiplexer by a corresponding transistor. The demultiplexer 1103 is configured so that each output line (shown as #1 through #32 in FIG. 11) provides an activation voltage signal (shown as signals B1,B1′ through B16,B16′) at the occurrence of a corresponding count 1112 of the counter 1102 received while the demultiplexer is enabled. Thus, the count 1112 of the counter 1102 provides the rate at which the stages of capacitor pairs 834a,834b are discharged. For instance, when the demultiplexer 1103 is enabled by the timer 1104, the first count of the counter 1102 may correspond to activation voltage signals (B1 and B1′) being applied to the first output (line #1) for the demultiplexer 1103, which in turn turns on respective transistors 834a,834b and discharges respective capacitors 830a,830b in the first stage (stage 1) of the charge accumulator 803. The second count of the counter 1102 may correspond to an activation voltage signals (B2 and B2′) being applied to the second output (line #2) for the demultiplexer 1103, which in turn turns on respective transistors 834a,834b and discharges respective capacitors 830a,830b in the second stage (stage 2) of the charge accumulator 803. This continues until all counts corresponding to all output lines (lines 1-16) and discharging of all capacitors 830a,830b of in all stages (stages 1-16) of charge accumulator 803 have occurred. After all stages have been discharged, timer 1104 may be configured to disable the demultiplexer 1103. For example, timer 1104 may be configured to receive the last activation signal corresponding to the discharge of the last stage and disable upon receipt, thus disabling the demultiplexer.

The RF tissue modulation devices may be configured to deliver RF energy from the RF energy source to the plasma generator for a therapeutic duration. The therapeutic duration may range, for example, from minutes or less, including 30 seconds or less, such as 10 seconds or less. In some instances, the therapeutic duration may range from 1 to 2 seconds. The therapeutic duration may be controlled using a variety of implementations. For example, the RF tissue modulation device may be configured to return switches 831, 835, 1107 to their charging positions after a predetermined amount of time.

When switches 831, 835, 1107 are returned to charging positions, the charge accumulator 803 may once again store charge in the capacitors 830. In some instances, the RF tissue modulation device is configured to recharge the charge accumulator within a minimum recharge period between plasma generation. The minimum recharge period may range, for example, from 10 minutes or less, including 5 minutes or less, such as 3 minutes or less. In some instances, the minimum recharge period ranges from 1 to 2 minutes. Various recharge periods can be implemented by varying, for example, battery size, voltage boosting levels, and/or capacitance sizes.

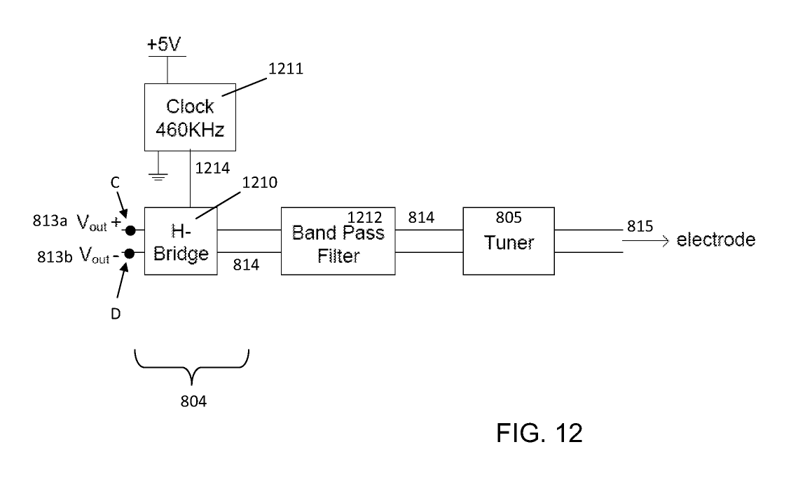

FIG. 12 illustrates a functional block diagram of an RF signal generator 804 and RF tuner 805 shown in FIG. 8, according to one embodiment. RF signal generator 804 outputs a high voltage modulated RF signal 814 at a specific operating frequency. In the embodiment shown, RF signal generator 804 includes an H-bridge 1210, an RF clock source 1211, and optional bandpass filter 1212. The H-bridge 1210 is coupled to the charge accumulator 803 and includes input lines at Point C and Point D that receive the positive and negative high voltage modulated signals 813a,813, respectively, provided by the positive and negative high voltage output rails at Point C and Point D, respectively, of charge accumulator 803 in FIG. 10 (when switch 835 is positioned accordingly).

H-bridge 1210 is coupled to an RF clock source 1211 and receives the positive and negative high voltage modulated signals 813a,813b at Point C and Point D. H-bridge 1210 switches the polarities of the positive and negative high voltage modulated signals 813a,813b based on an RF clock signal 1214 received by the RF clock source 1211, thus outputting a high voltage modulated RF signal 814. The switching provided at the output of the H-bridge 1210 is switched at an operating frequency based on the RF clock signal 1214. The operating frequency can range, for example, from 1 KHz to 50 MHz, such as from 100 KHz to 25 MHz, and including from 250 KHz to 10 MHz. In some embodiments, the RF voltage signal is a sine wave with operating frequency 460 kHz.

The resulting high voltage modulated RF signal 814 is provided to the plasma generator and provides the necessary power and voltage to generate a plasma. An optional bandpass filter 1212 is shown coupled to H-bridge 1210 and filters the signal to eliminate noise and output it to optional RF tuner 805. RF tuner 805 receives the high voltage modulated RF signal 814 and outputs a tuned high voltage modulated RF signal 815 as described above for FIG. 6.

### Methods

Aspects of the subject invention also include methods of modifying an internal target tissue of a subject. In certain embodiments, the methods of modifying an internal target site include positioning the distal end of a minimally invasive RF tissue modulation device at a target tissue site. In some instances, the RF tissue modulation device may comprise a hand-held control unit and RF probe, as described above. In some instances, the RF tissue modulation device may include an RF probe, medical device, and adapter operably coupled to the medical device.

The methods further include activating RF energy for delivery to a plasma generator at a distal end of the minimally invasive RF tissue modulation device. Still further, the methods include generating RF energy, delivering the RF energy to the plasma generator, and generating a plasma at the plasma generator to deliver RF energy to the internal target tissue site of the subject. For example, a plasma may be generated between an RF electrode of the plasma generator and the outer surface of the elongated member, resulting in tissue modification. In some instances, irrigating conducting fluid is provided. In some instances, the plasma generator may further be translated and/or rotated while supplying RF energy (and irrigating conducting fluid in some instances)—e.g, resulting in tissue dissection. In some instances, the entire end of the RF tissue modulation device may be translated proximally and distally until the desired tissue dissection is obtained. When finished with tissue dissection at the first location, the device may be rotated 180 degrees and further tissue removed using the steps described above.

Aspects of the subject invention may also include methods of generating RF energy for delivery to an internal target tissue of a subject. In some embodiments, the methods of generating RF energy include providing electrical energy from an electrical energy source to a charge accumulator, and storing energy in a charge accumulator. The methods may further include discharging the electrical energy to an RF signal generator and generating a modulated RF signal output. The methods may further include boosting the voltage of the modulated RF signal using a voltage converter to generate a high voltage modulated RF signal. In some instances, the methods further include providing the high voltage modulated RF signal to an RF tuner and outputting a tuned high voltage RF signal to a plasma generator.

In some embodiments, the methods of generating RF energy include providing electrical energy from an electrical energy source to a charge accumulator, storing energy in a charge accumulator, and discharging the electrical energy to voltage converter. The methods may further include providing an RF clock signal from an RF clock source to the voltage converter and generating a modulated high voltage signal output. The methods may further include providing the modulated high voltage signal to an RF signal generator to generate a high voltage modulated RF signal output. In some instances, the methods further include providing the high voltage modulated RF signal to an RF tuner and outputting a tuned high voltage RF signal to a plasma generator.

In some embodiments, the methods of generating RF energy include providing electrical energy from an electrical energy source to a voltage converter. The methods further include generating a high voltage positive and negative voltage, providing the high voltage positive and negative voltage to a charge accumulator, storing energy within the charge accumulator, and discharging positive and negative high voltage modulated signals from the charge accumulator. In some instances, the discharging of positive and negative high voltage modulated signals may include activating a modulation circuit to discharge the charge accumulator in stages at a modulated rate. The methods may further include providing the positive and negative high voltage modulated signals from the charge accumulator to an H-bridge operating at a frequency based on an RF clock signal to generate positive and negative high voltage modulated RF signal outputs. In some instances, the methods further include providing the high voltage modulated RF signal to an RF tuner and outputting a tuned high voltage RF signal to a plasma generator.

Aspects of the invention further include methods of imaging an internal tissue site with RF tissue modulation devices of the invention. A variety of internal tissue sites can be modified and/or imaged with devices of the invention. In certain embodiments, the methods are methods of imaging an intervertebral disc in a minimally invasive manner. For ease of description, the methods are now primarily described further in terms of imaging IVD target tissue sites. However, the invention is not so limited, as the devices may be used to image a variety of distinct target tissue sites.

With respect to imaging an intervertebral disc or portion thereof, e.g., exterior of the disc, nucleus pulposus, etc., embodiments of such methods include positioning a distal end of an RF tissue modulation device of the invention in viewing relationship to an intervertebral disc or portion of there, e.g., nucleus pulposus, internal site of nucleus pulposus, etc. By viewing relationship is meant that the distal end is positioned within 40 mm, such as within 10 mm, including within 5 mm of the target tissue site of interest. Positioning the distal end of the RF tissue modulation device in relation to the desired target tissue may be accomplished using any convenient approach, including through use of an access device, such as a cannula or retractor tube, which may or may not be fitted with a trocar, as desired.

Methods of invention may include visualizing the internal target tissue site via a visualization sensor integrated at the distal end of the elongated member of the RF tissue modulation device. The visualizing may include obtaining image data of an internal tissue site with the visualization sensor and then forwarding the image data to an image processing module of a system of the invention. Methods of invention may also include receiving image data into a system that includes an image processing module of the invention. The methods may further include viewing an image produced from the image data received by the image processing module. In some instances, the methods include visualizing the internal target tissue via a remote monitor.

Methods of the invention may further include illuminating the internal target tissue site via an illuminator integrated at the distal end of the elongated member. For example, following positioning of the distal end of the RF tissue modulation device in viewing relationship to the target tissue, the target tissue, e.g., intervertebral disc or portion thereof, is imaged through use of the illumination and visualization elements to obtain image data. Image data obtained according to the methods of the invention is output to a user in the form of an image, e.g., using a monitor or other convenient medium as a display means. In certain embodiments, the image is a still image, while in other embodiments the image may be a video.

In certain embodiments, the methods include a step of tissue modification using RF energy, as described in the methods above. For example, the methods may include a step of tissue removal using RF energy, e.g., using a combination of tissue cutting and irrigation or flushing. For example, the methods may include cutting a least a portion of the tissue using RF energy and then removing the cut tissue from the site, e.g., by flushing at least a portion of the imaged tissue location using a fluid introduced by an irrigation lumen and removed by an aspiration lumen.

The internal target tissue site may vary widely. Internal target tissue sites of interest include, but are not limited to, cardiac locations, vascular locations, orthopedic joints, central nervous system locations, etc. In certain cases, the internal target tissue site comprises spinal tissue.

The subject methods are suitable for use with a variety of mammals. Mammals of interest include, but are not limited to: race animals, e.g. horses, dogs, etc., work animals, e.g. horses, oxen etc., and humans. In some embodiments, the mammals on which the subject methods are practiced are humans.

Aspects of the invention further include methods of assembling an RF tissue modulation device. In these embodiments, the methods include operably coupling a proximal end of an elongated member to a hand-held control unit, e.g., as described above. Depending on the particular configuration, this step of operably coupling may include a variety of different actions, such as snapping the elongated member into a receiving structure of the hand-held control unit, twist locking the elongated member into a receiving structure of the hand-held control unit, and the like. In some instances, methods of assembling may further include sealing the hand-held control unit inside of a removable sterile covering, where the sterile covering is attached to the proximal end of the elongated member and configured to seal the hand-held control unit from the environment, e.g., as described above. In such instances, the methods may further include sealing a proximal end of the sterile covering.

In some embodiments, the methods of assembly include operably coupling a proximal end of an adapter to a hand-held medical device, e.g., a visualization device as described above. In some instances, the medical device includes a removable section that is removed before the adapter may be operably coupled. Depending on the particular configuration, this step of operably coupling may include a variety of different attachment mechanisms, such as snapping, hinging, using magnetics, etc. In some instances, medical device does not include a removable section that is required to be removed before operably coupling adapter to the medical device.

In some instances, methods of assembling may further include sealing the hand-held control unit inside of a removable sterile covering, where the sterile covering is attached to the proximal end of the elongated member and configured to seal the hand-held control unit from the environment, e.g., as described above. In such instances, the methods may further include sealing a proximal end of the sterile covering.

### Utility

The subject RF tissue modulation devices and methods find use in a variety of different applications where it is desirable to modify (and image, in some instances) an internal target tissue of a subject while minimizing damage to the surrounding tissue.

The subject devices and methods find use in many applications, such as but not limited to surgical procedures, that involve for example, removing small amounts of tissue via RF resection, RF ablation of a minor surface region of tissue, or coagulation of a limited area of exposed blood vessels, etc. Such surgical fields may include, for example, sports medicine, orthopedics, arthroscopy, spine surgery, laparoscopy, END, and neurosurgery. Example applications may include, for instance, debriding a torn meniscus, performing a micro-discectomy on a herniated lumbar disc, treating carpal tunnel syndrome by severing tissue around the nerve, etc.

The subject devices and methods find use in many applications, such as but not limited to surgical procedures, where a variety of different types of tissues may be removed, including but not limited to: soft tissue, cartilage, bone, ligament, etc. Specific procedures of interest include, but are not limited to, spinal fusion (such as Transforaminal Lumbar lnterbody Fusion (TLIF)), total disc replacement (TDR), partial disc replacement (PDR), procedures in which all or part of the nucleus pulposus is removed from the intervertebral disc (IVD) space, arthroplasty, and the like. As such, methods of the invention also include treatment methods, e.g., where a disc is modified in some manner to treat an existing medical condition. Treatment methods of interest include, but are not limited to: annulotomy, nucleotomy, discectomy, annulus replacement, nucleus replacement, and decompression due to a bulging or extruded disc. Additional methods in which the RF tissue modulation devices may find use include those described in United States Published Application No. 20080255563.

In certain embodiments, the subject devices and methods facilitate the dissection of the nucleus pulposus while minimizing thermal damage to the surrounding tissue. In addition, the subject devices and methods can facilitate the surgeon's accessibility to the entire region interior to the outer shell, or annulus, of the IVD, while minimizing the risk of cutting or otherwise causing damage to the annulus or other adjacent structures (such as nerve roots) in the process of dissecting and removing the nucleus pulposus.

Furthermore, the subject devices and methods may find use in other procedures, such as but not limited to ablation procedures, including high-intensity focused ultrasound (HIFU) surgical ablation, cardiac tissue ablation, neoplastic tissue ablation (e.g. carcinoma tissue ablation, sarcoma tissue ablation, etc.), microwave ablation procedures, and the like. Yet additional applications of interest include, but are not limited to: orthopedic applications, e.g., fracture repair, bone remodeling, etc., sports medicine applications, e.g., ligament repair, cartilage removal, etc., neurosurgical applications, and the like.

### Kits

Also provided are kits for use in practicing the subject methods, where the kits may include one or more of the above devices, and/or components thereof, e.g., elongated members (RF probes), hand-held control units, adapters, sterile coverings, etc., as described above. For example, the kits may include one or more of the following: a hand-held device as described above, an adapter as described above, an RF probe as described above, and other types of probes, such as a visualization probe. The kits may further include other components, e.g., guidewires, access devices, fluid sources, etc., which may find use in practicing the subject methods. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

It should be understood that some of the techniques introduced above can be implemented by programmable circuitry programmed or configured by software and/or firmware, or they can be implemented entirely by special-purpose “hardwired” circuitry, or in a combination of such forms. Such special-purpose circuitry (if any) can be in the form of, for example, one or more application-specific integrated circuits (ASICS), programmable logic devices (PLDs), field-programmable gate arrays (FPGAs), etc. For example, various switches, timers, etc., may be implemented in software and/or firmware, or they can be implemented entirely by special-purpose “hardwired” circuitry.

Software or firmware implementing the techniques introduced herein may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing took, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc. The term “logic”, as used herein, can include, for example, special purpose hardwired circuitry, software and/or firmware in conjunction with programmable circuitry, or a combination thereof. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A minimally invasive RF tissue modulation device, the device comprising:
(a) a hand-held control unit comprising an electrical energy source; and
(b) an elongated member having a proximal end operably coupled to the hand-held control unit and a minimally-dimensioned distal end comprising a plasma generator;
wherein the device is configured to generate a plasma at the plasma generator for a therapeutic duration.

2. The device according to claim 1, wherein the device comprises a voltage converter, a charge accumulator and an RF signal generator collectively operably coupling the electrical energy source to the plasma generator.

3. The device according to claim 2, wherein the RF signal generator comprises a power amplifier and an RF clock source.

4. The device according to claim 3, wherein the RF signal generator is configured to receive a first signal from the charge accumulator and to output a second signal to the voltage converter.

5. The device according to claim 4, wherein the RF signal generator is configured to receive a clock signal from a second clock source and to output the second signal as a modulated signal based on the clock signal.

6. The device according to claim 3, wherein the voltage converter is configured to receive a first signal from the charge accumulator and to output a second signal to the RF signal generator.

7. The device according to claim 6, wherein the voltage converter is configured to receive a clock signal and to output the second signal as a modulated signal based on the clock signal.

8. The device according to claim 3, wherein the charge accumulator is configured to receive a first signal from the voltage converter and to output a second signal to the RF signal generator.

9. The device according to claim 1, wherein the electrical energy source comprises one or more batteries.

10. The device according to claim 2, wherein the voltage converter is a DC to DC converter.

11. The device according to claim 2, wherein the charge accumulator comprises a single capacitor.

12. The device according to claim 2, wherein the charge accumulator comprises a set of two or more capacitor pairs.

13. The device according to claim 12, wherein the device comprises a demultiplexer configured to produce a modulated signal output from the set.

14. The device according to claim 13, wherein each capacitor pair of the set is coupled to the demultiplexer by a transistor.

15. The device according to claim 14, wherein the transistor is a bipolar junction transistor.

16. The device according to claim 3, wherein the RF signal generator comprises a H-bridge.

17. The device according to claim 1, further comprising a band pass filter.

18. The device according to claim 1, further comprising a tuner.

19-32. (canceled)

33. A method of delivering RF energy to an internal target tissue site of a subject, the method comprising:
(a) positioning the distal end of an elongated member of a device according to claim 1 at the internal target tissue site of a subject; and
(b) generating a plasma from the plasma generator to deliver RF energy to the internal target tissue site of the subject.

34-37. (canceled)

38. An adapter comprising:
an electrical energy source; and
a voltage converter;
a charge accumulator; and
an RF signal generator.

39-76. (canceled)
